SEQUENCE LISTING (59 sequences)

SEQ ID NO: 1 - protein, 13 aa, Artificial Sequence
  Synthetic
  MISC_FEATURE (3)..(4): Xaa is any hydrophobic amino acid
  MISC_FEATURE (6)..(6): Xaa is M or L
  MISC_FEATURE (7)..(7): Xaa is any hydrophobic amino acid
  MISC_FEATURE (10)..(11): Xaa is any hydrophobic amino acid
LAXXLXXLLXXLL

SEQ ID NO: 2 - protein, 13 aa, Artificial Sequence
  Synthetic
  MISC_FEATURE (6)..(6): X is M or L
LAIFLXALLIVLL

SEQ ID NO: 3 - protein, 19 aa, Artificial Sequence
  Synthetic
  MISC_FEATURE (1)..(1): X is R or K
  MISC_FEATURE (2)..(2): X is any hydrophobic amino acid
  MISC_FEATURE (4)..(5): X is any hydrophobic amino acid
  MISC_FEATURE (8)..(9): X is any hydrophobic amino acid
  MISC_FEATURE (12)..(12): X is any hydrophobic amino acid
  MISC_FEATURE (15)..(16): X is any hydrophobic amino acid
  MISC_FEATURE (19)..(19): X is W, Y, or L
XXQXXLAXXLMXLLXXLLX

SEQ ID NO: 4 - protein, 25 aa, Artificial Sequence
  Synthetic
  MISC_FEATURE (1)..(1): X is R or K
  MISC_FEATURE (4)..(4): X  is any hydrophobic amino acid
  MISC_FEATURE (7)..(8): X  is any hydrophobic amino acid
  MISC_FEATURE (11)..(11): X  is any hydrophobic amino acid
  MISC_FEATURE (14)..(15): X  is any hydrophobic amino acid
  MISC_FEATURE (18)..(18): X  is any hydrophobic amino acid
  MISC_FEATURE (21)..(22): X  is any hydrophobic amino acid
  MISC_FEATURE (25)..(25): X  is W, Y or L
XLSXSLXXQLXLAXXLMXLLXXLLX

SEQ ID NO: 5 - protein, 15 aa, Artificial Sequence
  Synthetic
  MISC_FEATURE (1)..(1): X is R or K
  MISC_FEATURE (4)..(5): X is any hydrophobic amino acid
  MISC_FEATURE (8)..(8): X is any hydrophobic amino acid
  MISC_FEATURE (11)..(12): X is any hydrophobic amino acid
  MISC_FEATURE (15)..(15): X is W, Y, or L
XLAXXLMXLLXXLLX

SEQ ID NO: 6 - protein, 19 aa, Artificial Sequence
  Synthetic
  MISC_FEATURE (1)..(1): X is R or K
  MISC_FEATURE (2)..(2): X is any hydrophobic amino acid
  MISC_FEATURE (5)..(5): X is any hydrophobic amino acid
  MISC_FEATURE (8)..(9): X is any hydrophobic amino acid
  MISC_FEATURE (12)..(12): X is any hydrophobic amino acid
  MISC_FEATURE (15)..(16): X is any hydrophobic amino acid
  MISC_FEATURE (19)..(19): X is W, Y, or L
XXQLXLAXXLLXLLXXLLX

SEQ ID NO: 7 - protein, 25 aa, Artificial Sequence
  Synthetic
  MISC_FEATURE (1)..(1): X is R or K
  MISC_FEATURE (4)..(4): X is any hydrophobic amino acid
  MISC_FEATURE (7)..(8): X is any hydrophobic amino acid
  MISC_FEATURE (11)..(11): X is any hydrophobic amino acid
  MISC_FEATURE (14)..(15): X is any hydrophobic amino acid
  MISC_FEATURE (18)..(18): X is any hydrophobic amino acid
  MISC_FEATURE (21)..(22): X is any hydrophobic amino acid
XLSXSLXXQLXLAXXLLXLLXXLLW

SEQ ID NO: 8 - protein, 29 aa, Artificial Sequence
  Synthetic
  MISC_FEATURE (1)..(1): X is R or K
  MISC_FEATURE (4)..(4): X is any hydrophobic amino acid
  MISC_FEATURE (7)..(8): X is any hydrophobic amino acid
  MISC_FEATURE (11)..(11): X is any hydrophobic amino acid
  MISC_FEATURE (14)..(15): X is any hydrophobic amino acid
  MISC_FEATURE (18)..(18): X is any hydrophobic amino acid
  MISC_FEATURE (21)..(22): X is any hydrophobic amino acid
  MISC_FEATURE (25)..(25): X is any hydrophobic amino acid
  MISC_FEATURE (28)..(28): X is any hydrophobic amino acid
  MISC_FEATURE (29)..(29): X is Y, W, or L
XLSXSLXXQLXLAXXLLXLLXXLLXLLXX

SEQ ID NO: 9 - protein, 25 aa, Artificial Sequence
  Synthetic
RLSFSLLLQLVLAIFLMALLIVLLW

SEQ ID NO: 10 - protein, 15 aa, Artificial Sequence
  Synthetic
RLAIFLMALLIVLLW

SEQ ID NO: 11 - protein, 19 aa, Artificial Sequence
  Synthetic
RLQLVLAIFLLALLIVLLW

SEQ ID NO: 12 - protein, 25 aa, Artificial Sequence
  Synthetic
RLSFSLLLQLVLAIFLLALLIVLLW

SEQ ID NO: 13 - protein, 29 aa, Artificial Sequence
  Synthetic
RLSFSLLLQLVLAIFLLALLIVLLVLLIY

SEQ ID NO: 14 - protein, 15 aa, Artificial Sequence
  Synthetic
RLAIFLMALLIVLLW

SEQ ID NO: 15 - protein, 15 aa, Artificial Sequence
  Synthetic
  MISC_FEATURE (1)..(1): X is any hydrophobic amino acid
  MISC_FEATURE (3)..(3): X is L or V
  MISC_FEATURE (4)..(5): X is any hydrophobic amino acid
  MISC_FEATURE (7)..(7): X is L or M
  MISC_FEATURE (8)..(8): X is any hydrophobic amino acid
  MISC_FEATURE (11)..(12): X is any hydrophobic amino acid
  MISC_FEATURE (14)..(14): X is V or I
  MISC_FEATURE (15)..(15): X is any hydrophobic amino acid
XLXXXIXXLVXXIXX

SEQ ID NO: 16 - protein, 15 aa, Artificial Sequence
  Synthetic
  MISC_FEATURE (1)..(1): X is Y or A
  MISC_FEATURE (3)..(3): X is L or V
  MISC_FEATURE (5)..(5): X is I or V
  MISC_FEATURE (7)..(7): X is L or M
  MISC_FEATURE (14)..(14): X is V or I
  MISC_FEATURE (15)..(15): X is A or R
XLXIXIXVLVLVIXX

SEQ ID NO: 17 - protein, 21 aa, Artificial Sequence
  Synthetic
  MISC_FEATURE (1)..(1): X is W, T, Q, or Y
  MISC_FEATURE (4)..(5): X is any hydrophobic amino acid
  MISC_FEATURE (8)..(8): X is any hydrophobic amino acid
  MISC_FEATURE (11)..(12): X is any hydrophobic amino acid
  MISC_FEATURE (15)..(15): X is any hydrophobic amino acid
  MISC_FEATURE (18)..(18): X is any hydrophobic amino acid
  MISC_FEATURE (19)..(19): X is is any polar amino acid
  MISC_FEATURE (21)..(21): X is K or R
XLLXXILXLVXXIVXLAXXQX

SEQ ID NO: 18 - protein, 25 aa, Artificial Sequence
  Synthetic
  MISC_FEATURE (1)..(1): X is W, T, Q, or Y
  MISC_FEATURE (4)..(5): X is any hydrophobic amino acid
  MISC_FEATURE (7)..(8): X is any hydrophobic amino acid
  MISC_FEATURE (11)..(12): X is any hydrophobic amino acid
  MISC_FEATURE (15)..(15): X is any hydrophobic amino acid
  MISC_FEATURE (18)..(19): X is any hydrophobic amino acid
  MISC_FEATURE (21)..(21): X is any hydrophobic amino acid
  MISC_FEATURE (22)..(22): X is any polar amino acid
  MISC_FEATURE (25)..(25): X is R or K
XLLXXIXXLVXXIVXLAXXQXXLVX

SEQ ID NO: 19 - protein, 15 aa, Artificial Sequence
  Synthetic
  MISC_FEATURE (1)..(1): X is W, T, Q, or Y
  MISC_FEATURE (4)..(5): X is any hydrophobic amino acid
  MISC_FEATURE (7)..(8): X is any hydrophobic amino acid
  MISC_FEATURE (11)..(12): X is any hydrophobic amino acid
  MISC_FEATURE (15)..(15): X is R or K
XLLXXIXXLVXXIVX

SEQ ID NO: 20 - protein, 21 aa, Artificial Sequence
  Synthetic
  MISC_FEATURE (1)..(1): X is W, T, Q, or Y
  MISC_FEATURE (4)..(5): X is any hydrophobic amino acid
  MISC_FEATURE (8)..(8): X is any hydrophobic amino acid
  MISC_FEATURE (11)..(12): X is any hydrophobic amino acid
  MISC_FEATURE (15)..(15): X is any hydrophobic amino acid
  MISC_FEATURE (18)..(18): X is any hydrophobic amino acid
  MISC_FEATURE (19)..(19): X is any polar amino acid
  MISC_FEATURE (21)..(21): X is R or K
XLVXXIMXLVXXIIXLAXXQX

SEQ ID NO: 21 - protein, 25 aa, Artificial Sequence
  Synthetic
  MISC_FEATURE (1)..(1): X is W, T, Q, or Y
  MISC_FEATURE (4)..(5): X is any hydrophobic amino acid
  MISC_FEATURE (8)..(8): X is any hydrophobic amino acid
  MISC_FEATURE (11)..(12): X is any hydrophobic amino acid
  MISC_FEATURE (15)..(15): X is any hydrophobic amino acid
  MISC_FEATURE (18)..(19): X is any hydrophobic amino acid
  MISC_FEATURE (22)..(22): X is any polar amino acid
  MISC_FEATURE (23)..(24): X is any hydrophobic amino acid
  MISC_FEATURE (25)..(25): X is R or K
XLVXXIMXLVXXIIXLAXXQMXXXX

SEQ ID NO: 22 - protein, 32 aa, Artificial Sequence
  Synthetic
  MISC_FEATURE (1)..(1): X is W, T, Q  orY
  MISC_FEATURE (4)..(5): X is any hydrophobic amino acid
  MISC_FEATURE (8)..(8): X is any hydrophobic amino acid
  MISC_FEATURE (11)..(12): X is any hydrophobic amino acid
  MISC_FEATURE (15)..(15): X is any hydrophobic amino acid
  MISC_FEATURE (18)..(19): X is any hydrophobic amino acid
  MISC_FEATURE (22)..(22): X is any hydrophobic amino acid
  MISC_FEATURE (25)..(26): X is any hydrophobic amino acid
  MISC_FEATURE (29)..(29): X is any polar amino acid
  MISC_FEATURE (32)..(32): X is R or K
XLVXXIVXLVXXIMXLVXXIIXLAXXQMXLVX

SEQ ID NO: 23 - protein, 21 aa, Artificial Sequence
  Synthetic
  MISC_FEATURE (1)..(1): X is W, T, Y, or Q
  MISC_FEATURE (4)..(5): X is any hydrophobic amino acid
  MISC_FEATURE (7)..(8): X is any hydrophobic amino acid
  MISC_FEATURE (11)..(12): X is any hydrophobic amino acid
  MISC_FEATURE (15)..(15): X is any hydrophobic amino acid
  MISC_FEATURE (18)..(18): X is any hydrophobic amino acid
  MISC_FEATURE (19)..(19): X is any polar amino acid
  MISC_FEATURE (21)..(21): X is R or K
XLLXXIXXLVXXIVXLAXXQX

SEQ ID NO: 24 - protein, 21 aa, Artificial Sequence
  Synthetic
YLLIVILVLVLVIVALAVTQK

SEQ ID NO: 25 - protein, 25 aa, Artificial Sequence
  Synthetic
YLLIVILVLVLVIVALAVLQLYLVR

SEQ ID NO: 26 - protein, 15 aa, Artificial Sequence
  Synthetic
YLLIVILVLVLVIVR

SEQ ID NO: 27 - protein, 21 aa, Artificial Sequence
  Synthetic
YLVIIIMVLVLVIIALAVTQK

SEQ ID NO: 28 - protein, 25 aa, Artificial Sequence
  Synthetic
YLVIIIMVLVLVIIALAVLQMYLVR

SEQ ID NO: 29 - protein, 32 aa, Artificial Sequence
  Synthetic
WLVIVIVALVIIIMVLVLVIIALAVLQMYLVR

SEQ ID NO: 30 - protein, 25 aa, Artificial Sequence
  Synthetic
  MISC_FEATURE (1)..(1): X is R or K
  MISC_FEATURE (4)..(4): X is any hydrophobic amino acid
  MISC_FEATURE (7)..(8): X is any hydrophobic amino acid
  MISC_FEATURE (11)..(11): X is any hydrophobic amino acid
  MISC_FEATURE (14)..(15): X is any hydrophobic amino acid
  MISC_FEATURE (18)..(18): X is any hydrophobic amino acid
  MISC_FEATURE (21)..(22): X is any hydrophobic amino acid
  MISC_FEATURE (25)..(25): X is W, Y, or L
XLSXSLXXQLXLAXXLLXLLXXLLX

SEQ ID NO: 31 - protein, 19 aa, Artificial Sequence
  Synthetic
  MISC_FEATURE (1)..(1): X is R or K
  MISC_FEATURE (2)..(2): X is any polar amino acid
  MISC_FEATURE (4)..(5): X is any hydrophobic amino acid
  MISC_FEATURE (8)..(9): X is any hydrophobic amino acid
  MISC_FEATURE (11)..(13): X is any hydrophobic amino acid
  MISC_FEATURE (15)..(16): X is any hydrophobic amino acid
  MISC_FEATURE (19)..(19): X is Y or W
XXIXXLLXXAXXXSXXIWX

SEQ ID NO: 32 - protein, 20 aa, Artificial Sequence
  Synthetic
  MISC_FEATURE (1)..(1): X is R or K
  MISC_FEATURE (2)..(2): X is any polar amino acid
  MISC_FEATURE (5)..(6): X is any hydrophobic amino acid
  MISC_FEATURE (8)..(9): X is any hydrophobic amino acid
  MISC_FEATURE (12)..(13): X is any hydrophobic amino acid
  MISC_FEATURE (15)..(17): X is any hydrophobic amino acid
  MISC_FEATURE (20)..(20): X is Y or W
XXIWXXIXXLLXXAXXXSZX

SEQ ID NO: 33 - protein, 19 aa, Artificial Sequence
  Synthetic
RTIMLLLVFAILLSAIIWY

SEQ ID NO: 34 - protein, 20 aa, Artificial Sequence
  Synthetic
RTIWIIIMLLLVFAILLSQY

SEQ ID NO: 35 - protein, 23 aa, Artificial Sequence
  Synthetic
  MISC_FEATURE (5)..(5): X is any hydrophobic amino acid
  MISC_FEATURE (12)..(12): X is any hydrophobic amino acid
  MISC_FEATURE (16)..(16): X is any hydrophobic amino acid
  MISC_FEATURE (23)..(23): X is R or K
TLLSXQLLLIAXMLVXIALLLSX

SEQ ID NO: 36 - protein, 19 aa, Artificial Sequence
  Synthetic
  MISC_FEATURE (1)..(1): X Is Q, W, T, or Y
  MISC_FEATURE (8)..(8): X  is any hydrophobic amino acid
  MISC_FEATURE (12)..(12): X  is any hydrophobic amino acid
  MISC_FEATURE (19)..(19): X  is R or K
XQLLLIAXMLVXIALLLSX

SEQ ID NO: 37 - protein, 23 aa, Artificial Sequence
  Synthetic
TLLSMQLLLIALMLVVIALLLSR

SEQ ID NO: 38 - protein, 19 aa, Artificial Sequence
  Synthetic
QQLLLIALMLVVIALLLSR

SEQ ID NO: 39 - protein, 19 aa, Artificial Sequence
  Synthetic
  MISC_FEATURE (1)..(1): X is R or K
  MISC_FEATURE (4)..(4): X is any hydrophobic amino acid
  MISC_FEATURE (8)..(8): X is any hydrophobic amino acid
  MISC_FEATURE (11)..(11): X is any hydrophobic amino acid
  MISC_FEATURE (15)..(15): X is any hydrophobic amino acid
  MISC_FEATURE (18)..(18): X is any hydrophobic amino acid
  MISC_FEATURE (19)..(19): X is W, Y  or L
XLLXAVAXLQXLNIXLVXX

SEQ ID NO: 40 - protein, 19 aa, Artificial Sequence
  Synthetic
KLLIAVALLQLLNILLVML

SEQ ID NO: 41 - protein, 19 aa, Artificial Sequence
  Synthetic
  MISC_FEATURE (1)..(1): X is W, T, Q, or Y
  MISC_FEATURE (4)..(5): X is any hydrophobic amino acid
  MISC_FEATURE (7)..(9): X is any hydrophobic amino acid
  MISC_FEATURE (11)..(12): X is any hydrophobic amino acid
  MISC_FEATURE (15)..(16): X is any hydrophobic amino acid
  MISC_FEATURE (18)..(18): X is any hydrophobic amino acid
  MISC_FEATURE (19)..(19): X is R or K
XMIXXVXXXSXXIVXXAXX

SEQ ID NO: 42 - protein, 19 aa, Artificial Sequence
  Synthetic
WMIVIVMFLSLAIVIVALR

SEQ ID NO: 43 - protein, 76 aa, Artificial Sequence
  Synthetic
MTRTEIIRELERSLRLQLVLAIFLMALLIVLLWLQQNGSSNNNVNYLLIVILVLVLVIVALAVTQKYLVEQLKRQD

SEQ ID NO: 44 - protein, 76 aa, Artificial Sequence
  Synthetic
MTSTYIITRLSFSLLLQLVLAIFLMALLIVLLWLQQNGSSNNNVNYLLIVILVLVLVIVALAVLQLYLVRQLHTQM

SEQ ID NO: 45 - protein, 76 aa, Artificial Sequence
  Synthetic
MTSTYIITRLSYSLREQLRLAIFLMALLIVLLWLQQNGSSNNNVNYLLIVILVLVLVIVRLAKEQKYLVEQLHTQM

SEQ ID NO: 46 - protein, 104 aa, Artificial Sequence
  Synthetic
MTRTEIIRELERSLRLQLVLAIFLLALLIVLLWLLQQLKELLRELERLQREGSSDEDVRELLREIKELVENIVYLVIIIMVLVLVIIALAVTQKYLVEELKRQD

SEQ ID NO: 47 - protein, 104 aa, Artificial Sequence
  Synthetic
MTRTEIITRLSFSLLLQLVLAIFLLALLIVLLWLLQQLKELLRELERLQREGSSDEDVRELLREIKELVENIVYLVIIIMVLVLVIIALAVLQMYLVRELKRQD

SEQ ID NO: 48 - protein, 104 aa, Artificial Sequence
  Synthetic
MTRTEIITRLSFSLLLQLVLAIFLLALLIVLLVLLIYLKELLRELERLQREGSSDEDVRELLREIKWLVIVIVALVIIIMVLVLVIIALAVLQMYLVRELKRQD

SEQ ID NO: 49 - protein, 156 aa, Artificial Sequence
  Synthetic
MTRTEIIRELERSLRLQLVLAIFLMALLIVLLWLQQNGSSNNNVNYLLIVILVLVLVIVALAVTQKYLVEQLKRQADPTDDSRTEIIRELERSLRLQLVLAIFLMALLIVLLWLQQNGSSNNNVNYLLIVILVLVLVIVALAVTQKYLVEQLKRQD

SEQ ID NO: 50 - protein, 86 aa, Artificial Sequence
  Synthetic
MSEELRAVADLQRLNIELARKLLIAVALLQLLNILLVMLTSELTDEKTILWMIVIVMFLSLAIVIVALREIRRAKEESRKIADESR

SEQ ID NO: 51 - protein, 70 aa, Artificial Sequence
  Synthetic
  misc_feature (7)..(7): Xaa can be any naturally occurring amino acid
MSKDTEXSRKIWRTIMLLLVFAILLSAIIWYQITTNPDTSQIATLLSMQLLLIALMLVVIALLLSRQTEQ

SEQ ID NO: 52 - protein, 215 aa, Artificial Sequence
  Synthetic
MSKDTEDSRKIWRTIMLLLVFAILLSAIIWYQITTNPDTSQIATLLSMQLLLIALMLVVIALLLSRQTEQVAESIRRDVSALAYVMLGLLLSLLNRLSLAAEAYKKAIELDPNDALAWLLLGSVLEKLKRLDEAAEAYKKAIELKPNDASAWKELGKVLEKLGRLDEAAEAYKKAIELDPEDAEAWKELGKVLEKLGRLDEAAEAYKKAIELAND

SEQ ID NO: 53 - protein, 87 aa, Artificial Sequence
  Synthetic
MGSKDTEDSRKIWRTIMLLLVFAILLSAIIWYQITQLLEEARKKGVSPVGAAEMLVQIATLLSMQLLLIALMLVVIALLLSRQTEQR

SEQ ID NO: 54 - protein, 217 aa, Artificial Sequence
  Synthetic
MGSKDTEDSRTIWIIIMLLLVFAILLSQYIWSQITTNPDTSQIATLLSQQLLLIALMLVVIALLLSRQTEQVAESIRRDVSALAYVMLGLLLSLLNRLSLAAEAYKKAIELDPNDALAWLLLGSVLEKLKRLDEAAEAYKKAIELKPNDASAWKELGKVLEKLGRLDEAAEAYKKAIELDPEDAEAWKELGKVLEKLGRLDEAAEAYKKAIELDPND

SEQ ID NO: 55 - protein, 216 aa, Artificial Sequence
  Synthetic
MSKDTEDSRTIWIIIMLLLVFAILLSQYIWSQITYNPDTSQIATLLSQQLLLIALMLVVIALLLSRQTEQVAESIRRDVSALAYVMLGLLLSLLNRLSLAAEAYKKAIELDPNDALAWLLLGSVLEKLKRLDEAAEAYKKAIYLKPNDASAWKELGKVLEKLGRLDEAAEAYKKAIELDPEDAEAWKELGKVLEKLGRLYEAAEAYKKAIELDPND

SEQ ID NO: 56 - protein, 231 aa, Artificial Sequence
  Synthetic
MGSKDTEDSRKIWRTIMLLLVFAILLSAIIWYQITQLLEEARKKGVSPVGAAEMLVQIATLLSMQLLLIALMLVVIALLLSRQTEQVAESIRRDVSALAYVMLGLLLSLLNRLSLAAEAYKKAIELDPNDALAWLLLGSVLEKLKRLDEAAEAYKKAIELKPNDASAWKELGKVLEKLGRLDEAAEAYKKAIELDPEDAEAWKELGKVLEKLGRLDEAAEAYKKAIELAND

SEQ ID NO: 57 - protein, 70 aa, Artificial Sequence
  Synthetic
MSKDTEDSRKIWEDIRRLLEEARKNSEEIWKEITKNPDTSEIARLLSEQLLEIAEMLVRIAELLSRQTEQ

SEQ ID NO: 58 - protein, 66 aa, Artificial Sequence
  Synthetic
MTRTEIIRELERSLREQEELARKLKELLRELERLQREGSSDEDVRELLREIKELVEEIEKELKRQD

SEQ ID NO: 59 - protein, 86 aa, Artificial Sequence
  Synthetic
MSEELRAVADLQRLNIELARKLLEAVARLQELNIDLVRKTSELTDEKTIREEIRKVKEESKRIVEEAEEEIRRAKEESRKIADESR

(19) United States
(12) Patent Application Publication
(10) Pub. No.: US 2021/0363214 A1
(43) Pub. Date: Nov. 25, 2021

(54) Transmembrane polypeptides

(71) Applicant: UNIVERSITY OF WASHINGTON, Seattie, WA (US)

(72) Inventors: Peilong LU, Seattle, WA (US); David BAKER, Seattle, WA (US); Scott BOYKEN, Seattle, WA (US); Zibo CHEN, Seattle, WA (US); Jorge FALLAS, Seattle, WA (US); George UEDA, Seattle, WA (US); William H. SHEFFLER, Seattle, WA (US)

(21) Appl. No.: 16/976,350

(22) PCT Filed: Feb. 28, 2019
(86) PCT No.: PCT/US2019/019948

§ 371 Date: Aug. 27, 2020

Related U.S. Application Data
(60) Provisional application No. 62/637,289, filed on Mar. 1, 2018.

(51) Int. Cl. C07K 14/705

(52) U.S. Cl. CPC C07K 14/705; C07K 2319/03

(57) ABSTRACT

De novo designed multi-pass transmembrane polypeptides are described, that include 2 or more transmembrane domains that are each between 15 and 35 amino acids in length, include one or more polar residues, and include at least 60%, 65%, 70%, 75%, 80%, 85%, 90%, or more hydrophobic amino acid residues.

### CROSS REFERENCE

This application claims priority to U.S. Provisional Patent Application Ser. No. 62/637,289 filed Mar. 1, 2018, incorporated by reference herein in its entirety.

### BACKGROUND

Design of transmembrane proteins with more than one membrane spanning region remains a major challenge. A major challenge for membrane protein design stems from the similarity of the membrane environment to protein hydrophobic cores. In the design of soluble proteins, the secondary structure and overall topology can be specified by the pattern of hydrophobic and hydrophilic residues, with the former inside the protein and the latter outside facing solvent. This core design principle cannot be used for membrane proteins, as the apolar environment of the hydrocarbon core of the lipid bilayer requires that outward facing residues in the membrane also be nonpolar.

### SUMMARY

In one aspect the disclosure provides non-naturally occurring polypeptide comprising the general formula X1-TM1-X2-TM2-X3, wherein

X1 is an optional first peptide domain

TM1 is a first transmembrane peptide of between 15 and 35 amino acids in length and capable of spanning a biological membrane, wherein (a) the first residue of TM1 is R or K; (b) the last residue of TM1 is W, Y, or L; and (c) at least 60%, 65%, 70%, 75%, 80%, 85%, 90%, or more of the internal residues are hydrophobic;

X2 comprises a first connecting peptide;

TM2 is a second transmembrane peptide of between 15 and 35 amino acids in length and capable of spanning a biological membrane, wherein (a) the first residue of TM2 is W, T, Q, or Y; (b) the last residue of TM2 is R or K; and (c) at least 60%, 65%, 70%, 75%, 80%, 85%, 90%, or more of the internal residues are hydrophobic; and

X3 is an optional second peptide domain;

wherein TM1 includes at least a first interior polar amino acid residue that is capable of forming a hydrogen bond with a first interior polar amino acid residue present in TM2. In various embodiments, TM1 and TM2 each include at least two or three interior polar amino acid residues capable of hydrogen bonding with interior amino acids of the other TM domain. In one embodiment, TM1 and TM2 are each between 15 and 32 amino acid residues in length. In another embodiment, the number of amino acid residues on TM1 and TM2 differ by 4 amino acids, 3 amino acids, 2 amino acids, 1 amino acid, or the number of amino acid residues in TM1 and TM2 are the same. In one embodiment, TM1 comprises the internal amino acid sequence LAXXL(M/L)XLLXXLL (SEQ ID NO: 1), wherein “X” is any hydrophobic amino acid. In another embodiment, TM 1 comprises the internal amino acid sequence LAIFL(M/L)ALLIVLL (SEQ ID NO:2). In various further embodiments, TM1 comprises the amino acid sequence selected from the group consisting of SEQ ID NOS: 3-14 wherein “X” is any hydrophobic amino acid:

In one embodiment, TM2 comprises the amino acid sequence XL(L/V)XXI(L/M)XLVXXI(V/I)X (SEQ ID NO: 15), wherein X is any hydrophobic amino acid. In another embodiment, TM2 comprises the amino acid sequence (Y/A)L(L/V)I(V/I)I(L/M)VLVLVI(V/I)(A/R) (SEQ ID NO: 16). In further embodiments, TM2 comprises the amino acid sequence selected from the group consisting of SEQ ID NOS: 17-2923 wherein X is any hydrophobic amino acid, and Z is any polar amino acid:

In various further embodiments, TM1 and TM2 comprise a pair selected from the group consisting of:

(a) TM1 comprises the amino acid sequence (R/K)XQXXLAXXLMXLLXXLL(W/Y/L) (SEQ ID NO: 3) and TM2 comprises the amino acid sequence (W/T/Q/Y)LLXXILXLVXXIVXLAXZQ(K/R) (SEQ ID NO: 17) (TMHC2);

(b) TM1 comprises the amino acid sequence (R/K)LSXSLXXQLXLAXXLMXLLXXLL(W/Y/L) (SEQ ID NO: 4) and TM2 comprises the amino acid sequence (W/T/Q/Y)LLXXIXXLVXXIVXLAXXQXZLV(R/K) (SEQ ID NO: 18) (TMHC2_L);

(c) TM1 comprises the amino acid sequence (R/K)LAXXLMXLLXXLL(W/Y/L) (SEQ ID NO: 5) and TM2 comprises the amino acid sequence (W/T/Q/Y)LLXXIXXLVXXIV(R/K) (SEQ ID NO: 19) (TMHC2_S);

(d) TM1 comprises the amino acid sequence (R/K)XQLXLAXXLLXLLXXLL(W/Y/L) (SEQ ID NO: 6) and TM2 comprises the amino acid sequence (W/T/Q/Y)LVXXIMXLVXXIIXLAXZQ(K/R) (SEQ ID NO: 20) (TMHC2_E);

(e) TM1 comprises the amino acid sequence (R/K)LSXSLXXQLXLAXXLLXLLXXLL(WNV/L) (SEQ ID NO: 30) and TM2 comprises the amino acid sequence (W/T/Q/Y)LVXXIMXLVXXIIXLAXXQMZXX(R/K) (SEQ ID NO: 21) (TMHC2_E_V1);

(f) TM1 comprises the amino acid sequence (R/K)LSXSLXXQLXLAXXLLXLLXXLLXLLX(Y/W/L) (SEQ ID NO: 8) and TM2 comprises the amino acid sequence (W/T/Q/Y)LVXXIVXLVXXIMXLVXXIIXLAXXQMZLV(R/K) (SEQ ID NO: 22) (TMHC2_E_V2); and

(g) TM1 comprises the amino acid sequence (R/K)XQXXLAXXLMXLLXXLL(W/Y/L) (SEQ ID NO: 3) and TM2 comprises the amino acid sequence (W/T/Q/Y)LLXXIXXLVXXIVXLAXZQ(K/R) (SEQ ID NO: 23);

wherein X is any hydrophobic amino acid and Z is any polar amino acid.

In still further embodiments, TM1 and TM2 comprise a pair selected from the group consisting of:

(a) TM 1 comprises the amino acid sequence RLQLVLAIFLMALLIVLLW (SEQ ID NO: 10) and TM2 comprises the amino acid sequence YLLIVLVLVLVIVALAVTQK (SEQ ID NO: 24) (TMHC2);

(b) TM 1 comprises the amino acid sequence RLSFSLLLQLVLAIFLMALLIVLLW (SEQ ID NO: 9) and TM2 comprises the amino acid sequence YLLIVILVLVLVIVALAVLQLYLVR (SEQ ID NO: 25) (TMHC2_L);

(c) TM 1 comprises the amino acid sequence RLAIFLMALLIVLLW (SEQ ID NO: 14) and TM2 comprises the amino acid sequence YLLIVILVLVLVIVR (SEQ ID NO: 26) (TMHC2_S);

(d) TM 1 comprises the amino acid sequence RLQLVLAIFLLALLIVLLW (SEQ ID NO: 11) and TM2 comprises the amino acid sequence YLVIIIMVLVLVIIALAVTQK (SEQ ID NO: 27) (TMHC2_E);

(e) TM 1 comprises the amino acid sequence RLSFSLLLQLVLAIFLLALLIVLLW (SEQ ID NO: 12) and TM2 comprises the amino acid sequence YLVIIIMVLVLVIIALAVLQMYLVR (SEQ ID NO: 28) (TMHC2_E_V1);

(f) TM 1 comprises the amino acid sequence RLSFSLLLQLVLAIFLLALLIVLLVLLIY(SEQ ID NO: 13) and TM2 comprises the amino acid sequence WLVIVIVALVIIIMVLVLVIIALAVLQMYLVR (SEQ ID NO: 29) (TMHC2_E_V2); and

(g) TM 1 comprises the amino acid sequence RLQLVLAIFLMALLIVLLW (SEQ ID NO: 10) and TM2 comprises the amino acid sequence YLLIVILVLVLVIVALAVTQK (SEQ ID NO: 24) (TMHC2_E_V2);

In another embodiment, the polypeptide is of the general formula X1-TM1-X2-TM2-X3-TM3-X4-TM4, wherein

X3 is a second connecting peptide;

TM3 is a third transmembrane peptide of between 15 and 35 amino acids in length and capable of spanning a biological membrane, wherein (a) the first residue of TM3 is R or K; (b) the last residue of TM3 is W, Y, or L; and (c) at least 60%, 65%, 70%, 75%, 80%, 85%, 90%, or more of the internal residues are hydrophobic;

X4 is an optional third connecting peptide; and

TM4 is an optional fourth transmembrane peptide of between 15 and 35 amino acids in length and capable of spanning a biological membrane, wherein (a) the first residue of TM4 is W, T, Q, or Y; (b) the last residue of TM4 is R or K; and (c) at least 60%, 65%, 70%, 75%, 80%, 85%, 90%, or more of the internal residues are hydrophobic.

In various embodiments, TM3 comprises the amino acid sequence of any embodiment of TM1 disclosed herein, and/or TM4 comprises the amino acid sequence of any embodiment of TM2 disclosed herein.

In another embodiment, TM1 comprises the amino acid sequence selected from the group consisting of SEQ ID NOS 31-34 wherein “X” is any hydrophobic amino acid and Z is any polar amino acid:

In a further embodiment, TM2 comprises the amino acid sequence selected from the group consisting of SEQ ID NOS: 35-38 wherein “X” is any hydrophobic amino acid

In another embodiment TM1 and TM2 comprise a pair selected from the group consisting of:

(a) TM1 comprises the amino acid sequence (R/K)ZIXXLLXXAXXXSXXIW(Y/W) (SEQ ID NO: 31) and TM2 comprises the amino acid sequence TLLSXQLLLIAXMLVXIALLLS(R/K) (SEQ ID NO: 35) (TMHC4)

(b) TM1 comprises the amino acid sequence (R/K)ZIXXLLXXAXXXSXXIW(Y/W) (SEQ ID NO: 31) and TM2 comprises the amino acid sequence TLLSXQLLLIAXMLVXIALLLS(R/K) (SEQ ID NO: 35) (TMHC4_R)

(c) TM1 comprises the amino acid sequence (R/K)ZIXXLLXXAXXXSXXIW(Y/W) (SEQ ID NO: 31) and TM2 comprises the amino acid sequence TLLSXQLLLIAXMLVXIALLLS(R/K) (SEQ ID NO: 35) (TMHC4_E)

(d) TM1 comprises the amino acid sequence (R/K)ZIWXXIXXLLXXAXXXSZ(Y/W) (SEQ ID NO: 32) and TM2 comprises the amino acid sequence (Q/W/T/Y)QLLLIAXMLVXIALLLS(R/K) (SEQ ID NO: 36) (TMHC4_R_V1)

(e) TM1 comprises the amino acid sequence (R/K)ZIWXXIXXLLXXAXXXSZ(Y/W) (SEQ ID NO: 32) and TM2 comprises the amino acid sequence (Q/W/T/Y)QLLLIAXMLVXIALLLS(R/K) (SEQ ID NO: 36) (TMHC4_R_V2)

(f) TM1 comprises the amino acid sequence (R/K)ZIXXLLXXAXXXSXXIW(Y/W) (SEQ ID NO: 31) and TM2 comprises the amino acid sequence TLLSXQLLLIAXMLVXIALLLS(R/K) (SEQ ID NO: 35) (TMHC4_R_V3);

wherein X is any hydrophobic amino acid.

In another embodiment, TM1 and TM2 comprise a pair selected from the group consisting of:

(a) TM1 comprises the amino acid sequence RTIMLLLVFAILLSAIIWY (SEQ ID NO: 33) and TM2 comprises the amino acid sequence TLLSMQLLLIALMLWIALLLSR (SEQ ID NO: 37) (TMHC4)

(b) TM1 comprises the amino acid sequence RTIMLLLVFAILLSAIIWY (SEQ ID NO: 33) and TM2 comprises the amino acid sequence TLLSMQLLLIALMLWIALLLSR (SEQ ID NO: 37) (TMHC4_R)

(c) TM1 comprises the amino acid sequence RTIMLLLVFAILLSAIIWY (SEQ ID NO: 33) and TM2 comprises the amino acid sequence TLLSMQLLLIALMLWIALLLSR (SEQ ID NO: 37) (TMHC4_E)

(d) TM1 comprises the amino acid sequence RTIWIIIMLLLVFAILLSQY (SEQ ID NO: 34) and TM2 comprises the amino acid sequence QQLLLIALMLWIALLLSR (SEQ ID NO: 38) (TMHC4_R_V1)

(e) TM1 comprises the amino acid sequence RTIWIIIMLLLVFAILLSQY (SEQ ID NO: 34) and TM2 comprises the amino acid sequence QQLLLIALMLWIALLLSR (SEQ ID NO: 38) (TMHC4_R_V2); and

(f) TM1 comprises the amino acid sequence RTIMLLLVFAILLSAIIWY (SEQ ID NO: 33) and TM2 comprises the amino acid sequence TLLSMQLLLIALMLWIALLLSR (SEQ ID NO: 37) (TMHC4_R_V3).

In another embodiment, TM1 comprises the amino acid sequence of SEQ ID NO: 39 or 40, wherein X is any hydrophobic amino acid: (R/K)LLXAVAXLQXLNIXLVX(W/Y/L) (SEQ ID NO: 39) KLLIAVALLQLLNILLVML (SEQ ID NO: 40).

In a further embodiment, TM2 comprises the amino acid sequence (W/T/Q/Y)MIXXVXXXSXXIVXXAX(R/K) (SEQ ID NO: 41) or WMIVIVMFLSLAIVIVALR (SEQ ID NO: 42), wherein X is any hydrophobic amino acid.

In one embodiment, TM1 comprises the amino acid sequence (R/K)LLXAVAXLQXLNIXLVX(W/Y/L) (SEQ ID NO: 39) and TM2 comprises the amino acid sequence (W/T/Q/Y)MIXXVXXXSXXIVXXAX(R/K) (SEQ ID NO: 41), wherein X is any hydrophobic amino acid. In another embodiment, TM1 comprises KLLIAVALLQLLNILLVML (SEQ ID NO: 40) and TM2 comprises the amino acid sequence WMIVIVMFLSLAIVIVALR (SEQ ID NO:42).

In other embodiments the polypeptide is of the general formula X1-(TM1-X2-TM2-X3)ₙ, wherein n is 1, 2, 3, or 4.

In further embodiments, the polypeptide comprises the amino acid sequence at least 50%, 55%, 60%, 65%, 70%, 75%, 80%, 85%, 90%, 91%, 92%, 93%, 94%, 95%, 96%, 97%, 98%, 99%, or 100% identical along the length of the amino acid sequence selected from the group consisting of SEQ ID NOS: 43-56.

In one embodiment, the polypeptides may further comprise one or more bioactive polypeptide. In one such embodiment, the one or more bioactive polypeptide is present in the X1, X2, X3, or X4 domain, or wherein the one or more bioactive polypeptide is fused to the N-terminus or C-terminus of the polypeptide.

The disclosure also provides nucleic acids encoding the polypeptides of the disclosure, expression vectors comprising the nucleic acids of the disclosure operatively linked to a control sequence, host cells comprising the nucleic acids or the expression vectors of the disclosure, and uses of the polypeptides nucleic acids, expression vectors and the host cell of the disclosure.

### DESCRIPTION OF THE FIGURES

FIG. 1. Design and characterization of proteins with four transmembrane helices. From left to right, designs and data are shown for TMHC2 (transmembrane hairpin C2), TMHC2_E (elongated), TMHC2_L (long span) and TMHC2_S (short span). (A) Design models with intra- and extra-membrane regions with different lengths. Horizontal lines demarcate the hydrophobic membrane regions. Ribbon diagrams are on left, electrostatic surfaces on right, and the neutral transmembrane regions are in gray. (B) Representative analytical ultracentrifugation sedimentation-equilibrium curves at three different rotor speeds. Each data set is globally well fitted as a single ideal species in solution corresponding to the dimer molecular weight. ‘MW (D)’ and ‘MW (E)’ indicate the molecular weight of the oligomer design and that determined from experiment, respectively. (C) CD spectra and temperature melt (inset). No apparent unfolding transitions are observed up to 95° C.

FIG. 2. Folding stability of the 156-residue single chain TMHC2 (scTMHC2) design with four transmembrane helices. (A) Design model (left) and electrostatic surface (right) of scTMHC2. Numbers indicate the order of the four TMs in the sequence. Single-molecule forced unfolding experiments were conducted by applying mechanical tension to the N- and C-terminus of a single scTMHC2. (B) CD spectra of scTMHC2 at different temperatures. No unfolding transition is observed up to 95° C. (C) Single-molecule force-extension traces of scTMHC2. The unfolding and refolding transitions are denoted with arrows. (D) Folding energy landscape obtained from the single-molecule experiments. N, I, and U indicate the native, intermediate, and unfolded state respectively.

FIG. 3. Crystal structure of the designed transmembrane dimer TMHC2_E. (A and B) Crystal lattice packing. (A) The extended soluble region mediates a large portion of the crystal lattice packing. The TMs form layers in the crystal separating the soluble regions. (B) The C2 axis of the design aligns with the crystallographic two fold. Two monomers are paired in a dimer while the other two form two C2 dimers with two crystallographic adjacent monomers. The space group diagram (C121) is shown in the background. (C) Superposition of the TMHC2_E crystal structure and design model (RMSD=0.7 Å over the core Cα atoms). (D) The side-chain packing arrangements at layers (squares in panel C) at different depths in the membrane are almost identical to the design model.

FIG. 4. Stability and structural characterization of designs with six and eight membrane spanning helices. (A) Model of designed transmembrane trimer TMHC3 with six transmembrane helices. Stick representation from periplasmic side (left) and lateral surface view (right) are shown. (B) Circular dichroism characterization of TMHC3; the design is stable up to 95° C. (C) Representative analytical ultracentrifugation sedimentation-equilibrium curves at three different rotor speeds for TMHC3. The data fit to a single ideal species in solution with molecular weight close to that of the designed trimer. (D) Model of designed transmembrane tetramer TMHC4_R with eight transmembrane helices. (E) Analytical ultracentrifugation sedimentation-equilibrium curves at three different rotor speeds for TMHC4_R fit well to a single species with a measured molecular weight of ˜94 kDa. (F) Crystal structure of TMHC4_R. The overall tetramer structures are very similar to the design model, with a helical bundle body and helical repeat fins. The outer helices of the transmembrane hairpins tilt off the axis by ˜10°. (G) Cross section through the TMHC4_R crystal structure and electrostatic surface; the HRD forms a bowl at the base of the overall structure with a depth of ˜20 Å. The transmembrane region is indicated in lines. (H) Three views of the backbone superposition of TMHC4_R crystal structure and design model.

FIG. 5. Design sequences. Hydrophobic TMs are indicated above the sequences. (A) Sequence alignment of TMHC2 (SEQ ID NO: 43) with water-soluble version 2L4HC2_23 (SEQ ID NO: 58). (B) Sequence alignment of designed transmembrane dimers with different TMs lengths (SEQ ID NO: 43) (SEQ ID NO: 44) (SEQ ID NO: 45). (C) Sequence alignment of TMHC2 (SEQ ID NO: 43) with TMHC2_E (SEQ ID NO: 48). (D) Sequence of scTMHC2 (SEQ ID NO: 49). Sequence alignment of (E) TMHC3 (SEQ ID NO: 50) with 5L6HC3_1 (SEQ ID NO: 59) and (F) TMHC4_R TMs (SEQ ID NO: 51) with 5L8HC4_6 (SEQ ID NO: 57).

FIG. 6. Purification of designed multipass transmembrane proteins. (A) Representative gel filtration chromatography and SDS-PAGE of TMHC2, TMHC2_L and TMHC2_E. These dimeric designs elute at similar elution volume in gel filtration. TMHC2_L and TMHC2_E run at roughly dimer positions in SDS-PAGE. Only SDS-PAGE is shown for TMHC2_S, which expressed and behaved poorly. (B) Purification of scTMHC2. The elution volume of the major peak is comparable to the dimers. The small peak which elutes earlier is also from scTMHC2, probably due to intermolecular oligomers. Full separation of the two peaks is achieved after single chromatography. (C) Purification of TMHC3 trimer and TMHC4_R tetramer. TMHC3 runs at dimer position in SDS-PAGE, which may be an artifact due to incomplete denaturation.

FIG. 7. Refolding size analysis. (A) Example force-extension trace for refolding size analysis. The refolding step size to the intermediate state was measured at the point of a refolding event (red line). For comparison, the total refolding size was measured at the same force by measuring the extension difference between the fully unfolded and the full folded states (blue line). Notations N, 1, and U in the panel indicate the native, intermediate, and unfolded states respectively. (B) Scatter plot of extension size vs force. The values for intermediate refolding size (U to I) and the total refolding size (between N and U) are denoted with red and blue dots respectively (each N=166). (C) Count histogram for size ratio. The size ratio was calculated as the intermediate refolding size divided by the total refolding size. The histogram was fitted with Gaussian function (peak: 0.53, standard deviation: 0.08), indicating that half the protein is refolded in the intermediate state.

FIG. 8. Conceptual three-state energy landscape. (A) Energy landscape during unfolding at high force. The high force tilts the zero-force landscape toward the unfolded state so that during unfolding the main energy barrier is effectively reduced to the one between the native and intermediate states. (B) Energy landscape during refolding at low force. The landscape is slightly tilted at lower forces and the both energy barriers become prominent during refolding. Notations N, I, and U in the panels indicate the native, intermediate, and unfolded state respectively.

FIG. 9. Nearly identical structures for the three dimers in the crystal of TMHC2_E. (A) Structures for the three TMHC2_E dimers. Monomers those shown in FIG. 3B. (B) Structure alignment for the three dimers with Cα RMSDs between 0.60 and 0.84 Å.

FIG. 10. Sampling the helical junction between helical bundle 5L8HC4_6 and helical repeat homo-tetramer tpr1C4_2. Three successive views of junction assemblies. The ensemble of inserted helical linker and helical repeat domain is shown moving relative to the helical bundle as a result of sampling the helical linker. The tetramer structure of the helical repeat domain kept intact with defined tetrameric distance constraints.

FIG. 11. Crystal lattice packing for TMHC4_R. The helical repeat domain mediates a major portion of the crystal lattice packing of the 4 tetramers. There is no direct crystal contacts from transmembrane helical bundle, however, detergents may mediate some contacts between helical bundle and helical repeat domains.

FIG. 12. Structural analysis for TMHC4_R. (A) Structure alignments for the four monomers (left) and tetramers (right). The four monomers and tetramers could be aligned with Cα RMSDs from 0.2 to 0.6 Å and 0.2 to 1.0 Å, respectively. (B) Superpositions of crystal structure and design model for the TMHC4_R monomer. Structure alignments of the transmembrane, linker and HR domains are shown on the left, while the overall structure superposition is on the right. (C) The crystallographic four fold aligns with the C4 axis of the design. The space group diagram (P4) is shown in the background. (D) Structure alignments of crystal structure and design model for the TMHC4_R tetramer. The overall tetramer structure aligns to the design with Cα RMSDs of 3.3-3.8 Å (left). The first 162 residues of the tetramer in crystal structure align to the design with Cα RMSDs of 2.2-2.3 Å (right).

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise. “And” as used herein is interchangeably used with “or” unless expressly stated otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

In one aspect the disclosure provides non-naturally occurring polypeptides comprising the general formula X1-TM1-X2-TM2-X3, wherein

X1 is an optional first peptide domain

TM1 is a first transmembrane peptide of between 15 and 35 amino acids in length and capable of spanning a biological membrane, wherein (a) the first residue of TM1 is R or K; (b) the last residue of TM1 is W. Y, or L; and (c) at least 60%, 65%, 70%, 75%, 80%, 85%, 90%, or more of the internal residues are hydrophobic;

X2 comprises a first connecting peptide;

TM2 is a second transmembrane peptide of between 15 and 35 amino acids in length and capable of spanning a biological membrane, wherein (a) the first residue of TM2 is W, T, Q, or Y; (b) the last residue of TM2 is R or K; and (c) at least 60%, 65%, 70%, 75%, 80%, 85%, 90%, or more of the internal residues are hydrophobic; and

X3 is an optional second peptide domain;

wherein TM1 includes at least a first interior polar amino acid residue that is capable of forming a hydrogen bond with a first interior polar amino acid residue present in TM2.

As disclosed in the examples that follow, the inventors have designed a variety of transmembrane polypeptides containing 2-4 membrane spanning regions that adopt the target oligomerization state in detergent solution. Thus, the disclosure provides a significant advance in the design of transmembrane proteins with more than one membrane spanning region. Such polypeptides can be used for any suitable purpose, including but not limited to displaying antigens on membranes (for example, as a vaccine), as membrane localization markers, and/or as a stable scaffold to stabilize a target protein.

The polypeptides include at least 2 transmembrane domains (TM1 and TM2), and may contain any additional number of transmembrane domains as deemed appropriate for a given use (i.e.: TM3, TM4, TM5, TM6, etc.).

Each transmembrane peptide is capable of spanning a biological membrane and is between 15 and 35 amino acids in length; in other embodiments, each TM domain may be 15-34, 15-33, 15-32, 15-31, 15-30, 15-29, 15-28, 15-27, 15-26, 15-25, 15-24, 15-23, 15-22, 15-21, 15-20, 15-19, 15-18, 15-17, 15-16, 16-35, 16-34, 16-33, 16-32, 16-31, 16-30, 16-29, 16-28, 16-27, 16-26, 16-25, 16-24, 16-23, 16-22, 16-21, 16-20, 16-19, 16-18, 16-17, 17-35, 17-34, 17-33, 17-32, 17-31, 17-30, 17-29, 17-28, 17-27, 17-26, 17-25, 17-24, 17-23, 17-22, 17-21, 17-20, 17-19, 17-18, 18-35, 18-34, 18-33, 18-32, 18-31 18-30, 18-29, 18-28, 18-27, 18-26, 18-25, 18-24, 18-23, 18-22, 18-21, 18-20, 18-19, 19-35, 19-34, 19-33, 19-32, 19-31, 19-30, 19-29, 19-28, 19-27, 19-26, 19-25, 19-24, 19-23, 19-22, 19-21, 19-20, 20-35, 20-34, 20-33, 20-32, 20-31, 20-30, 20-29, 20-28, 20-27, 20-26, 20-25, 20-24, 20-23, 20-22, 20-21, 21-35, 21-34, 21-33, 21-32, 21-31, 21-30, 21-29, 21-28, 21-27, 21-26, 21-25, 21-24, 21-23, 21-22, 22-35, 22-34, 22-33, 22-32, 22-31, 22-30, 22-29, 22-28, 22-27, 22-26, 22-25, 22-24, 22-23, 23-35, 23-34, 23-33, 23-32, 23-31, 23-30, 23-29, 23-28, 23-27, 23-26, 23-25, 23-24, 24-35, 24-34, 24-33, 24-32, 24-31, 24-30, 24-29, 24-28, 24-27, 24-26, 24-25, 25-35, 25-34, 25-33, 25-32, 25-31, 25-30, 25-29, 25-28, 25-27, 25-26, 26-35, 26-34, 26-33, 26-32, 26-31, 26-30, 26-29, 26-28, 26-27, 27-35, 27-34, 27-33, 27-32, 27-31, 27-30, 27-29, 27-28, 28-35, 28-34, 28-33, 28-32, 28-31, 28-30, 28-29, 29-35, 29-34, 29-33, 29-32, 29-31, 29-30, 30-35, 30-34, 30-33, 30-32, 30-31, 31-35, 31-34, 31-33, 31-32, 32-35, 32-34, 32-33, 33-35, 33-34, 34-35, 15, 16, 17, 18, 19, 20, 21, 22, 23, 24, 25, 26, 27, 28, 29, 30, 31, 32, 33, 34, or 35 amino acids in length.

TM1 has (a) a first residue of R or K; (b) a last residue of W, Y, or L; and (c) at least 60%, 65%, 70%, 75%, 80%, 85%, 90%, or more of the internal residues (i.e.: all residues that are not the first or last residue in the TM domain) are hydrophobic.

TM2 has (a) a first residue of W, T, Q, or Y; (b) a last residue of R or K; and (c) at least 60%, 65%, 70%, 75%, 80%, 85%, 90%, or more of the internal residues are hydrophobic. As used herein, hydrophobic amino acid residues include Ala (A), Ile (I), Leu (L), Val (V), Met (M), and Phe (F).

TM1 and TM2 further include at least one interior polar amino acid residue that are capable of forming a hydrogen bond with each other. In various embodiments, TM1 and TM2 each include at least 2 or 3 interior polar amino acid residues capable of hydrogen bonding with one or more interior amino acids of the other TM domain. As used herein, polar amino acid residues include Gln (Q), Ser (S), Thr (T), Tyr (Y), Trp (V, Asn (N), and His (H). In specific embodiments, the polar amino acid residues include Gin (Q), Ser (S), Thr (T), Tyr (Y), and/or Trp (W).

In various embodiments, TM1 and TM2 differ in amino acid residue number by no more than 4, 3, 2, or 1 amino acid. In a further embodiment, the number of amino acid residues in TM1 and TM2 are identical.

In one embodiment, TM1 comprises the internal amino acid sequence LAXXL(M/L)XLLXXLL (SEQ ID NO: 1), wherein “X” is any hydrophobic amino acid and the residues in parentheses are optional amino acids that may be present at the position. This sequence is present in transmembrane proteins exemplified herein (i.e.: TMHC2 and its derivatives) that form homodimers via non-covalent bonding. In this embodiment, the residues in bold and underlined font are present as core resides in the TMCH2 polypeptides while the other residues are present on the surface and thus more readily modified. In a further embodiment, TM 1 comprises the internal amino acid sequence LAIFL(M/L)ALLIVLL (SEQ ID NO: 2).

In various further embodiments, TM1 comprises the amino acid sequence selected from the group consisting of those shown below, wherein “X” is any hydrophobic amino acid and the residues in parentheses are optional amino acids that may be present at the position. The amino acid sequence of the embodiments is the top line; the bottom line, consisting of “S” and “C” refers to surface (S) or core (C) residues present in the relevant polypeptide (this arrangement is continued throughout the disclosure). The surface residues can be modified to any hydrophobic amino acid.

```
TMHC2
(SEQ ID NO: 3)
(R/K)XQXXLAXXLMXLLXXLL(W/Y/L)
        SSCSSCCSSCCSCCSSCCS
TMHC2_L
(SEQ ID NO: 4)
(R/K)LSCSLXXQLXLAXXLMXLLXXLX(W/Y/L)
        SCCSCCSSCCSCCSSCCSCCSSCSS
TMHC2_S
(SEQ ID NO: 5)
(R/K)LAXXLMXLLXXLL(W/Y/L)
        SCCSSCCSCCSSCCS
TMHC2_E
(SEQ ID NO: 6)
(R/K)XQLXLAXXLLXLLXXLL(W/Y/L)
        SSCCSCCSSCCSCCSSCCS
TMHC2_E_V1
(SEQ ID NO: 7)
(R/K)LSXSLXXQLXLAXXLLXLLXXLLW
SCCSCCSSCCSCCSSCCSCCSSCCS
TMHC2_E_V2
(SEQ ID NO: 8)
(R/K)LSXSLSSQLXLAXXLLXLLXXLLXLLX(Y/W/L)
        SCCSCCSSCCSCCSSCCSCCSSCCSCCSS
scTMHC2
(SEQ ID NO: 3)
(R/K)XQXXLAXXLMXLLXXLL(W/Y/L)
        SSCSSCCSSCCSCCSSCCS
```

In various further embodiments, TM1 comprises the amino acid sequence selected from the group consisting of those shown below.

TMHC2 and scTMHC2
(SEQ ID NO: 10)
RLQLVLAIFLMALLIVLLW
SSCSSCCSSCCSCCSSCCS
RMHC2_L
(SEQ ID NO: 9)
RLSFSLLLQLVLAIFLMALLIVLLW
SCCSCCSSCCSCCSSCCSCCSSCSS
TMHC2_S
(SEQ ID NO: 14)
RLAIFLMALLIVLLW
SCCSSCCSCCSSCCS
TMHC2_E
(SEQ ID NO: 11)
RLQLVLAIFLLALLIVLLW
SSCCSCCSSCCSCCSSCCS
TMHC2_E_V1
(SEQ ID NO: 12)
RLSFSLLLQLVLAIFLLALLIVLLW
SCCSCCSSCCSCCSSCCSCCSSCCS
TMHC2_E_V2
(SEQ ID NO: 13)
RLSFSLLLQLVLAIFLLALLIVLLVLLIY
SCCSCCSSCCSCCSSCCSCCSSCCSCCSS

In various further embodiments, TM2 comprises the amino acid sequence XL(L/V)XXI(L/M)XLVXXI(V/I)X (SEQ ID NO: 15), wherein X is any hydrophobic amino acid and the residues in parentheses are optional amino acids that may be present at the position. This sequence is present in dimeric transmembrane proteins exemplified herein (i.e.: TMHC2 and its derivatives). In a further embodiment, TM2 comprises the amino acid sequence (Y/A)L(L/V)I(V/I)I(L/M)VLVLVI(V/I)(A/R) (SEQ ID NO: 16). In further embodiments, TM2 comprises the amino acid sequence selected from the group shown below, wherein X is any hydrophobic amino acid, and Z is any polar amino acid.

```
TMHC2
(SEQ ID NO: 17)
(W/T/Q/Y)LLXXILXLVXXIVXLAXZQ(K/R)
                  SCCSSCCSCCSSCCSCCSSCS
TMHC2_L
(SEQ ID NO: 18)
(W/T/Q/Y)LLXXIXXLVXXIVXLAXXQXZLV(R/K)
                  SCCSSCSSCCSSCCSCCSSCSSCCS
TMHC2_S
(SEQ ID NO: 19)
(W/T/Q/Y)LLXXIXXLVXXIV(R/K)
                SCCSSCSSCCSSCCS
TMHC2_E
(SEQ ID NO: 20)
(W/T/Q/Y)LVXXIMXLVXXIIXLAXZQ(K/R)
                SCCSSCCSCCSSCCSCCSSCC
TMHC2_E_V1
(SEQ ID NO: 21)
(W/T/Q/Y)LVXXIMXLVXXIIXLAXXQMZXX(R/K)
                SCCSSCCSCCSSCCSCCSSCCSCCS
TMHC2_E_V2
(SEQ ID NO: 22)
(W/T/Q/Y)LVXXIVXLVXXIMXLVXXIIXLAXXQMZLV(R/K)
                SCCSSCCSCCSSCCSCCSSCCSCCSSCCSCCS
scTMHC2
(SEQ ID NO: 23)
(W/T/Q/Y)LLXXIXXLVXXIVXLAXZQ(K/R)
                SCCSSCSSCCSSCCSCCSSCS
```

In further embodiments, TM2 comprises the amino acid sequence selected from the group shown below.

TMHC2 and scTMHC2
(SEQ ID NO: 24)
YLLIVILVLVLVIVALAVTQK
SCCSSCCSCCSSCCSCCSSCS
TMHC2_L
(SEQ ID NO: 25)
YLLIVILVLVLVIVALAVLQLYLVR
SCCSSCSSCCSSCCSCCSSCSSCCS
TMHC2_S
(SEQ ID NO: 26)
YLLIVILVLVLVIVR
SCCSSCSSCCSSCCS
TMHC2_E
(SEQ ID NO: 27)
TLVIIIMVLVLVIIALAVTQK
SCCSSCCSCCSSCCSCCSSCC
TMHC2_E_V1
(SEQ ID NO: 28)
YLVIIIMVLVLVIIALAVLQMYLVR
SCCSSCCSCCSSCCSCCSSCCSCCS
TMHC2_E_V2
(SEQ ID NO: 29)
WLVIVIVALVIIIMVLVLVIIALAVLQMYLVR
SCCSSCCSCCSSCCSCCSSCCSCCSSCCSCCS

In another embodiment, TM1 and TM2 comprise a pair selected from the group consisting of:

(a) TM1 comprises the amino acid sequence (R/K)XQXXLAXXLMXLLXXLL(W/Y/L) (SEQ ID NO: 3) and TM2 comprises the amino acid sequence (W/T/Q/Y)LLXXILXLVXXIVXLAXZQ(K/R) (SEQ ID NO: 17) (TMHC2);

(b) TM1 comprises the amino acid sequence (R/K)LSXSLXXQLXLAXXLMXLLXXLX(W/Y/L) (SEQ ID NO: 4) and TM2 comprises the amino acid sequence (W/T/Q/Y)LLXXIXXLVXXIVXLAXXQXZLV(R/K) (SEQ ID NO: 18) (TMHC2_L);

(c) TM1 comprises the amino acid sequence (R/K)LAXXLMXLLXXLL(W/Y/L) (SEQ ID NO: 5) and TM2 comprises the amino acid sequence (W/T/Q/Y)LLXXIXXLVXXIV(R/K) (SEQ ID NO: 19) (TMHC2_S);

(d) TM1 comprises the amino acid sequence (R/K)XQLXLAXXLLXLLXXLL(W/Y/L) (SEQ ID NO: 6) and TM2 comprises the amino acid sequence (W/T/Q/N)LVXXIMXLVXXIIXLAXZQ(K/R) (SEQ ID NO: 20) (TMHC2_E);

(e) TM1 comprises the amino acid sequence (R/K)LSXSLXXQLXLAXXLLXLLXXLL(WNV/L) (SEQ ID NO: 30) and TM2 comprises the amino acid sequence (W/T/Q/Y)LVXXIMXLVXXIIXLAXXQMZXX(R/K) (SEQ ID NO: 21) (TMHC2_E_V1);

(f) TM1 comprises the amino acid sequence (R/K)LSXSLXXQLXLAXXLLXLLXXLLXLLX(Y/W/L) (SEQ ID NO: 8) and TM2 comprises the amino acid sequence (W/T/Q/Y)LVXXIVXLVXXIMXLVXXIIXLAXXQMZLV(R/K) (SEQ ID NO: 22) (TMHC2_E_V2); and

(g) TM1 comprises the amino acid sequence (R/K)XQXXLAXXLMXLLXXLL(W/Y/L) (SEQ ID NO: 3) and TM2 comprises the amino acid sequence (W/T/Q/Y)LLXXIXXLVXXIVXLAXZQ(K/R) (SEQ ID NO: 23);

wherein X is any hydrophobic amino acid and Z is any polar amino acid.

In a further embodiment, TM1 and TM2 comprise a pair selected from the group consisting of:

(a) TM 1 comprises the amino acid sequence RLQLVLAIFLMALLIVLLW (SEQ ID NO: 10) and TM2 comprises the amino acid sequence YLLIVILVLVLVIVALAVTQK (SEQ ID NO: 24) (TMHC2);

(b) TM 1 comprises the amino acid sequence RLSFSLLLQLVLAIFLMALLIVLLW (SEQ ID NO: 9) and TM2 comprises the amino acid sequence YLLIVILVLVLVIVALAVLQLYLVR (SEQ ID NO: 25) (TMHC2_L);

(c) TM 1 comprises the amino acid sequence RLAIFLMALLIVLLW (SEQ ID NO: 14) and TM2 comprises the amino acid sequence YLLIVILVLVLVIVR (SEQ ID NO: 26) (TMHC2_S);

(d) TM 1 comprises the amino acid sequence RLQLVLAIFLLALLIVLLW (SEQ ID NO: 11) and TM2 comprises the amino acid sequence YLVIIIMVLVLVIIALAVTQK (SEQ ID NO: 27) (TMHC2_E);

(e) TM 1 comprises the amino acid sequence RLSFSLLLQLVLAIFLLALLIVLLW (SEQ ID NO: 12) and TM2 comprises the amino acid sequence YLVIIIMVLVLVIIALAVLQMYLVR (SEQ ID NO: 28) (TMHC2_E_V1);

(f) TM 1 comprises the amino acid sequence RLSFSLLLQLVLAIFLLALLIVLLVLLIY (SEQ ID NO: 13) and TM2 comprises the amino acid sequence WLVIVIVALVIIIMVLVLVIIALAVLQMYLVR (SEQ ID NO: 29) (TMHC2_E_V2); and

(g) TM 1 comprises the amino acid sequence RLQLVLAIFLMALLIVLLW (SEQ ID NO: 10) and TM2 comprises the amino acid sequence YLLIVILVLVLVIVALAVTQK (SEQ ID NO: 24) (TMHC2_E_V2);

In a further embodiment, the polypeptide is of the general formula X1-TM1-X2-TM2-X3-TM3-X4-TM4, wherein

X3 is a second connecting peptide;

TM3 is a third transmembrane peptide of between 15 and 35 amino acids in length and capable of spanning a biological membrane, wherein (a) the first residue of TM3 is R or K; (b) the last residue of TM3 is W. Y, or L; and (c) at least 60%, 65%, 70%, 75%, 80%, 85%, 90%, or more of the internal residues are hydrophobic;

X4 is an optional third connecting peptide; and

TM4 is an optional fourth transmembrane peptide of between 15 and 35 amino acids in length and capable of spanning a biological membrane, wherein (a) the first residue of TM4 is W, T, Q, or Y; (b) the last residue of TM4 is R or K; and (c) at least 60%, 65%, 70%, 75%, 80%, 85%, 90%, or more of the internal residues are hydrophobic.

Each of TM3 and TM4 are capable of spanning a biological membrane and is between 15 and 35 amino acids in length; in other embodiments, TM3 and TM4 domains may be 15-34, 15-33, 15-32, 15-31, 15-30, 15-29, 15-28, 15-27, 15-26, 15-25, 15-24, 15-23, 15-22, 15-21, 15-20, 15-19, 15-18, 15-17, 15-16, 16-35, 16-34, 16-33, 16-32, 16-31, 16-30, 16-29, 16-28, 16-27, 16-26, 16-25, 16-24, 16-23, 16-22, 16-21, 16-20, 16-19, 16-18, 16-17, 17-35, 17-34, 17-33, 17-32, 17-31, 17-30, 17-29, 17-28, 17-27, 17-26, 17-25, 17-24, 17-23, 17-22, 17-21, 17-20, 17-19, 17-18, 18-35, 18-34, 18-33, 18-32, 18-31 18-30, 18-29, 18-28, 18-27, 18-26, 18-25, 18-24, 18-23, 18-22, 18-21, 18-20, 18-19, 19-35, 19-34, 19-33, 19-32, 19-31, 19-30, 19-29, 19-28, 19-27, 19-26, 19-25, 19-24, 19-23, 19-22, 19-21, 19-20, 20-35, 20-34, 20-33, 20-32, 20-31, 20-30, 20-29, 20-28, 20-27, 20-26, 20-25, 20-24, 20-23, 20-22, 20-21, 21-35, 21-34, 21-33, 21-32, 21-31, 21-30, 21-29, 21-28, 21-27, 21-26, 21-25, 21-24, 21-23, 21-22, 22-35, 22-34, 22-33, 22-32, 22-31, 22-30, 22-29, 22-28, 22-27, 22-26, 22-25, 22-24, 22-23, 23-35, 23-34, 23-33, 23-32, 23-31, 23-30, 23-29, 23-28, 23-27, 23-26, 23-25, 23-24, 24-35, 24-34, 24-33, 24-32, 24-31, 24-30, 24-29, 24-28, 24-27, 24-26, 24-25, 25-35, 25-34, 25-33, 25-32, 25-31, 25-30, 25-29, 25-28, 25-27, 25-26, 26-35, 26-34, 26-33, 26-32, 26-31, 26-30, 26-29, 26-28, 26-27, 27-35, 27-34, 27-33, 27-32, 27-31, 27-30, 27-29, 27-28, 28-35, 28-34, 28-33, 28-32, 28-31, 28-30, 28-29, 29-35, 29-34, 29-33, 29-32, 29-31, 29-30, 30-35, 30-34, 30-33, 30-32, 30-31, 31-35, 31-34, 31-33, 31-32, 32-35, 32-34, 32-33, 33-35, 33-34, 34-35, 15, 16, 17, 18, 19, 20, 21, 22, 23, 24, 25, 26, 27, 28, 29, 30, 31, 32, 33, 34, or 35 amino acids in length.

TM3 has (a) a first residue of R or K; (b) a last residue of W. Y, or L; and (c) at least 60%, 65%, 70%, 75%, 80%, 85%, 90%, or more of the internal residues (i.e.: all residues that are not the first or last residue in the TM domain) are hydrophobic.

TM4 has (a) a first residue of W, T, Q, or Y; (b) a last residue of R or K; and (c) at least 60%, 65%, 70%, 75%, 80%, 85%, 90%, or more of the internal residues are hydrophobic.

TM3 and TM4 further include at least one interior polar amino acid residue that are capable of forming a hydrogen bond with each other and/or with polar amino acids in TM1 and/or TM2. In various embodiments, TM3 and TM4 each include at least 2 or 3 interior polar amino acid residues capable of hydrogen bonding with one or more interior amino acids of one or more of the other TM domains. In specific embodiments, the polar amino acid residues include Gln (Q), Ser (S), Thr (T), Tyr (Y), and/or Trp (W).

In various embodiments, TM1 and TM2 differ in amino acid residue number by no more than 4, 3, 2, or 1 amino acid. In a further embodiment, the number of amino acid residues in TM1 and TM2 are identical.

In other embodiments, TM4 is present and X4 is present. In various embodiments, TM3 may comprise the amino acid sequence of any embodiment of TM1 disclosed herein, and/or TM4 may comprise the amino acid sequence of any embodiment of TM2 disclosed herein.

In one embodiment TM1 comprises the amino acid sequence selected from the group below, wherein W is any hydrophobic amino acid and Z is any polar amino acid. These sequences are present in transmembrane proteins exemplified herein (i.e.: TMHC4 and its derivatives) that may form homotetramers through non-covalent binding.

```
TMHC4, TMHC4_R, TMHC4_E, and TMHC4_R_V3
(SEQ ID NO: 31)
(R/K)ZIXXLLXXAXXXSXXIW(Y/W)
        SSCSSCCSSCSSSCSSCCS
TMHC4_R_V1 and TMHC4_R_V2
(SEQ ID NO: 32)
(R/K)ZIWXXIXXLLXXAXXXSZ(Y/W)
        SSCCSSCSSCCSSCSSSCSS
```

In another embodiment TM1 comprises the amino acid sequence selected from the group below, wherein WX is any hydrophobic amino acid and Z is any polar amino acid.

TMHC4
(SEQ ID NO: 33)
RTIMLLLVFAILLSAIIWY
SSCSSCCSSCSSSCSSCCS
TMHC4_R, TMHC4_E, and TMHC4_R_V3
(SEQ ID NO: 33)
RTIMLLLVFAILLSAIIWY
SSCSSCCSSCSSSCSSCCS
TMHC4_R_V1 and TMHC4_R_V2
(SEQ ID NO: 34)
RTIWIIIMLLLVFAILLSQY
SSCCSSCSSCCSSCSSSCSS

In a further embodiment of the transmembrane proteins exemplified herein that form homotetramers. TM2 comprises the amino acid sequence selected from the group below, wherein ‘X’ is any hydrophobic amino acid.

TMHC4
(SEQ ID NO: 35)
TLLSXQLLLIAXMLVXIALLLS(R/K)
CCCCSCCCCCCSCCCSCCCCCCS
TMHC4_R_V1
(SEQ ID NO: 36)
(Q/W/T/Y)QLLLIAXMLVXIALLLS(R/K)
SCCCCCCSCCCSCCCCCCS

In another embodiment, TM2 comprises an amino acid sequence shown below, wherein X is any hydrophobic amino acid, wherein ‘X’ is any hydrophobic amino acid.

TMHC4, TMHC4_R, TMHC4_E, and TMHC4_4_V3
(SEQ ID NO: 37)
TLLSMQLLLIALMLVVIALLLSR
CCCCSCCCCCCSCCCSCCCCCCS
TMHC4_R_V1 and TMHC4_R_V2
(SEQ ID NO: 38)
QQLLLIALMLVVIALLLSR
SCCCCCCSCCCSCCCCCCS

In further embodiments, TM1 and TM2 comprise a pair selected from the group consisting of:

(a) TM1 comprises the amino acid sequence (R/K)ZIXXLLXXAXXXSXXIW(Y/W) (SEQ ID NO: 31) and TM2 comprises the amino acid sequence TLLSXQLLLIAXMLVXIALLLS(R/K) (SEQ ID NO: 35) (TMHC4)

(b) TM1 comprises the amino acid sequence (R/K)ZIXXLLXXAXXXSXXIW(Y/W) (SEQ ID NO: 31) and TM2 comprises the amino acid sequence TLLSXQLLLIAXMLVXIALLLS(R/K) (SEQ ID NO: 35) (TMHC4_R)

(c) TM1 comprises the amino acid sequence (R/K)ZIXXLLXXAXXXSXXIW(Y/W) (SEQ ID NO: 31) and TM2 comprises the amino acid sequence TLLSXQLLLIAXMLVXIALLLS(R/K) (SEQ ID NO: 35) (TMHC4_E)

(d) TM1 comprises the amino acid sequence (R/K)ZIWXXIXXLLXXAXXXSZ(Y/W) (SEQ ID NO: 32) and TM2 comprises the amino acid sequence (Q/W/T/Y)QLLLIAXMLVXIALLLS(R/K) (SEQ ID NO: 36) (TMHC4_R_V1)

(e) TM1 comprises the amino acid sequence (R/K)ZIWXXIXXLLXXAXXXSZ(Y/W) (SEQ ID NO: 32) and TM2 comprises the amino acid sequence (Q/W/T/Y)QLLLIAXMLVXIALLLS(R/K) (SEQ ID NO: 36) (TMHC4_R_V2)

(f) TM1 comprises the amino acid sequence (R/K)ZIXXLLXXAXXXSXXIW(Y/W) (SEQ ID NO: 31) and TM2 comprises the amino acid sequence TLLSXQLLLIAXMLVXIALLLS(R/K) (SEQ ID NO: 35) (TMHC4_R_V3);

wherein X is any hydrophobic amino acid.

In other embodiments, TM1 and TM2 comprise a pair selected from the group consisting of:

(a) TM1 comprises the amino acid sequence RTIMLLLVFAILLSAIIWY (SEQ ID NO: 33) and TM2 comprises the amino acid sequence TLLSMQLLLIALMLWIALLLSR (SEQ ID NO: 37) (TMHC4)

(b) TM1 comprises the amino acid sequence RTIMLLLVFAILLSAIIWY (SEQ ID NO: 33) and TM2 comprises the amino acid sequence TLLSMQLLLIALMLWIALLLSR (SEQ ID NO: 37) (TMHC4_R)

(c) TM1 comprises the amino acid sequence RTIMLLLVFAILLSAIIWY (SEQ ID NO: 33) and TM2 comprises the amino acid sequence TLLSMQLLLIALMLVVIALLLSR (SEQ ID NO: 37) (TMHC4_E)

(d) TM1 comprises the amino acid sequence RTIWIIMLLLVFAILLSQY (SEQ ID NO: 34) and TM2 comprises the amino acid sequence QQLLLIALMLWIALLLSR (SEQ ID NO: 38) (TMHC4_R_V1)

(e) TM1 comprises the amino acid sequence RTIWIIMLLLVFAILLSQY (SEQ ID NO: 34) and TM2 comprises the amino acid sequence QQLLLIALMLWIALLLSR (SEQ ID NO: 38) (TMHC4_R_V2); and

(f) TM1 comprises the amino acid sequence RTIMLLLVFAILLSAIIWY (SEQ ID NO: 33) and TM2 comprises the amino acid sequence TLLSMQLLLIALMLWIALLLSR (SEQ ID NO: 37) (TMHC4_R_V3).

In another embodiment, TM1 comprises the amino acid sequence

(SEQ ID NO: 39)
(R/K)LLXAVAXLQXLNIXLVX(W/Y/L)
SCCSCCCSCCSCCCSCCSS

wherein X is any hydrophobic amino acid. This sequence is present in transmembrane proteins exemplified herein (i.e.: TMHC3) that form homotrimers through non-covalent binding. In one embodiment TM1 comprises the amino acid sequence KLLIAVALLQLLNILLVML (SEQ ID NO: 40). In another embodiment, TM2 comprises the amino acid sequence below, wherein X is any hydrophobic amino acid:

(SEQ ID NO: 41)
(W/T/Q/Y)MIXXVXXXSXXIVXXAX(R/K)
SCCSSCSSSCSSCCSSCSS.

In a further embodiment, TM2 comprises the amino acid sequence WMIVIVMFLSLAIVIVALR (SEQ ID NO: 42). In another embodiment TM1 comprises amino acid sequence (R/K)LLXAVAXLQXLNIXLVX(W/Y/L) (SEQ ID NO: 39) and TM2 comprises the amino acid sequence (W/T/Q/Y)MIXXVXXXSXXIVXXAX(R/K) (SEQ ID NO: 41), wherein X is any hydrophobic amino acid. In a further embodiment, TM1 comprises KLLIAVALLQLLNILLVML (SEQ ID NO: 40) and TM2 comprises the amino acid sequence WMIVIVMFLSLAIVIVALR (SEQ ID NO: 42).

In further embodiments of each of the embodiments disclosed above, the polypeptide is of the general formula X1-(TM1-X2-TM2-X3)ₙ, wherein n is 1, 2, 3, or 4.

In all of these embodiments the connecting peptide domains X1, X2, X3, and X4 may be of any suitable length and amino acid composition. These domains either serve as linker s between TM domains or as N- or C-terminal residues on the polypeptide, and thus may be modified as desired for any suitable purpose. Thus, for example, other functional domains may be inserted into X1, X2, X3, or X4 as appropriate for an intended use. In one embodiment, X2 is at least 7 amino acids in length. In various other embodiments, one or both of X1 and X3 are present and are at least 1 amino acid in length.

In other embodiments, the polypeptide comprises the amino acid sequence at least 50%, 55%, 60%, 65%, 70%, 75%, 80%, 85%, 90%, 91%, 92%, 93%, 94%, 95%, 96%, 97%, 98%, 99%, or 100% identical along the length of the amino acid sequence selected from the group consisting of the following (underlined and bold-faced residues are TM domains; the position of surface (S) and core (C) residues are noted below the amino acid sequence)

TMHC3
(SEQ ID NO: 50)
MSEELRAVADLQRLNIELARKLLIAVALLQLLNILLVMLTSELTDEKTI
LWMIVIVMFLSLAIVIVALREIRRAKEESRKIADESR
SSSCCSCCCSCCSCCCSCCSSCCSCCCSCCSCCCSCCSSCCSCSSSSSC
SSCCSSCSSSCSSCCSSCSSCCSSCCSSCSSCCSSCS
TMHC4
(SEQ ID NO: 51)
MSKDTEXSRKIWRTIMLLLVFAILLSAIIWYQITTNPDTSQIATLLSMQ
LLLIALMLVVIALLLSRQTEQ
SSSCSSSCSSCCSSCSSCCSSCSSSCSSCCSSCSSSSSCCSCCCCCCSC
CCCCCSCCCSCCCCCCSCCCS
TMHC4_R
(SEQ ID NO: 52)
MSKDTESSRKIWRTIMLLLVFAILLSAIIWYQITTNPDTSQIATLLSMQ
LLLIALMLVVIALLLSRQTEQVAESIRRDVSALAYVMLGLLLSLLNRLS
LAAEAYKKAIELDPNDALAWLLLGSVLEKLKRLDEAAEAYKKAIELKPN
DASAWKELGKVLEKLGRLDEAAEAYKKAIELDPEDAEAWKELGKVLEKL
GRLDEAAEAYKKAIELAND
SSSCCSSCSSCCSSCSSCCSSCSSSCSSCCSSCSSSSSCCSCCCCCCSC
CCCCCSCCCSCCCCCCSCCCCSCCCSSCSSCCCCCCCCCSCCCSSSCCS
SCCSCCSCCCSSCSSCCCCCCCCCSCCSSSSSSSSCSSCSSSCSSCCSS
TMHC4_E
(SEQ ID NO: 53)
MGSKDTEDSRKIWRTIMLLLVFAILLSAIIWYQITQLLEEARKKGVSPV
GAAEMLVQIATLLSMQLLLIALMLVVIALLLSRQTEQR
SSSSSSSSCSSCCSSCSSCCSSCSSSCSSCCSSCSSCCSSCSSSSCSCC
CCCCCCCSCCCCCCSCCCCCCSCCCSCCCCCCSCCCSS
TMHC4_R_V1
(SEQ ID NO: 54)
MGSKDTEDSRTIWIIIMLLLVFAILLSQYIWSQITTNPDTSQIATLLSQ
QLLLIALMLVVIALLLSRQTEQVAESIRRDVSALAYVMLGLLLSLLNRL
SLAAEAYKKAIELDPNDALAWLLLGSVLEKLKRLDEAAEAYKKAIELKP
NDASAWKELGKVLEKLGRLDEAAEAYKKAIELDPEDAEAWKELGKVLEK
LGRLDEAAEAYKKAIELDPND
SSSSCCSSCSSCCSSCSSCCSSCSSSCSSCCSSCSSSSSCCSCCCCCCS
CCCCCCSCCCSCCCCCCSCCCSCCCCCCCCCCCCCCCCCCCCCCSSSCC
CCCCCCCSSCCSCCSSCCCCCCCCCCCCCSCCCCSSCCSCCSCCCSSCS
SCCCCCCCCCSCCCSSSCCSSCCSCCSCCCSSCSSCCCCCCCCCSCCSS
SSSSSSCSSCSSSCSSCCSSS
TMHC4_R_V2
(SEQ ID NO: 55)
MSKDTEDSRTIWIIIMLLLVFAILLSQYIWSQITYNPDTSQIATLLSQQ
LLLIALMLVVIALLLSRQTEQVAESIRRDVSALAYVMLGLLLSLLNRLS
LAAEAYKKAIELDPNDALAWLLLGSVLEKLKRLDEAAEAYKKAIYLKPN
DASAWKELGKVLEKLGRLDEAAEAYKKAIELDPEDAEAWKELGKVLEKL
GRLYEAAEAYKKAIELDPND
SSSCCSSCSSCCSSCSSCCSSCSSSCSSCCSSCSSSSSCCSCCCCCCSC
CCCCCSCCCSCCCCCCSCCCSCCCCCCCCCCCCCCCCCCCCCCSSSCCC
CCCCCCSSCCSCCSSCCCCCCCCCCCCCSCCCCSSCCSCCSCCCSSCSS
CCCCCCCCCSCCCSSSCCSSCCSCCSCCCSSCSSCCCCCCCCCSCCSSS
SSSSSCSSCSSSCSSCCSSS
TMHC4_R_V3
(SEQ ID NO: 56)
MGSKDTEDSRKIWRTIMLLLVFAILLSAIIWYQITQLLEEARKKGVSPV
GAAEMLVQIATLLSMQLLLIALMLVVIALLLSRQTEQVAESIRRDVSAL
AYVMLGLLLSLLNRLSLAAEAYKKAIELDPNDALAWLLLGSVLEKLKRL
DEAAEAYKKAIELKPNDASAWKELGKVLEKLGRLDEAAEAYKKAIELDP
EDAEAWKELGKVLEKLGRLDEAAEAYKKAIELAND
SSSSSSSSCSSCCSSCSSCCSSCSSSCSSCCSSCSSCCSSCSSSSCSCC
CCCCCCCSCCCCCCSCCCCCCSCCCSCCCCCCSCCCSSCCCCCCCCCCC
CCCCCCCCCCSSSCCCCCCCCCSSCCSCCSSCCCCCCCCCCCCCSCCCC
SSCCSCCSCCCSSCSSCCCCCCCCCSCCCSSSCCSSCCSCCSCCCSSCS
SCCCCCCCCCSCCSSSSSSSSCSSCSSSCSSCCSS
TMHC2
(SEQ ID NO: 43)
MTRTEIIRELERSLRLQLVLAIFLMALLIVLLWLQQNGSSNNNVNYLLI
VILVLVLVIVALAVTQKYLVEQLKRQD
SSCSSCCSSCCSCCSSCSSCCSSCCSCCSSCCSCCSSSSSCSSCCSCCS
SCCSCCSSCCSCCSSCSSCCSSCCSSS
TMHC2_L
(SEQ ID NO: 44)
MTSTYIITRLSFSLLLQLVLAIFLMALLIVLLWLQQNGSSNNNVNYLLI
VILVLVLVIVALAVLQLYLVRQLHTQM
SSCSSCCSSCCSCCSSCCSCCSSCCSCCSSCSSCCSSSSSCSSCCSCCS
SCSSCCSSCCSCCSSCSSCCSSCCSSS
TMHC2_S
(SEQ ID NO: 45)
MTSTYIITRLSYSLREQLRLAIFLMALLIVLLWLQQNGSSNNNVNYLLI
VILVLVLVIVRLAKEQKYLVEQLHTQM
SSCSSCCSSCCSCCSSCCSCCSSCCSCCSSCCSCCSSSSSCSSCCSCCS
SCSSCCSSCCSCCSSCSSCCSSCCSSS
TMHC2_E
(SEQ ID NO: 46)
MTRTEIIRELERSLRLQLVLAIFLLALLIVLLWLLQQLKELLRELERLQ
REGSSDEDVRELLREIKELVENIVYLVIIIMVLVLVIIALAVTQKYLVE
ELKRQD
SSCSSCCSSCCSCCSSCCSCCSSCCSCCSSCCSCCSSCCSCCSSCCSCC
SSSSSSSSCSSCCSSCCSCCSSCCSCCSSCCSCCSSCCSCCSSCCSCCS
SCCSSS
TMHC2_E_V1
(SEQ ID NO: 47)
MTRTEIITRLSFSLLLQLVLAIFLLALLIVLLWLLQQLKELLRELERLQ
REGSSDEDVRELLREIKELVENIVYLVIIIMVLVLVIIALAVLQMYLVR
ELKRQD
SSCSSCCSSCCSCCSSCCSCCSSCCSCCSSCCSCCSSCCSCCSSCCSCC
SSSSSSSSCSSCCSSCCSCCSSCCSCCSSCCSCCSSCCSCCSSCCSCCS
SCCSSS
>TMHC2_E_V2
(SEQ ID NO: 48)
MTRTEIITRLSFSLLLQLVLAIFLLALLIVLLVLLIYLKELLRELERLQ
REGSSDEDVRELLREIKWLVIVIVALVIIIMVLVLVIIALAVLQMYLVR
ELKRQD
SSCSSCCSSCCSCCSSCCSCCSSCCSCCSSCCSCCSSCCSCCSSCCSCC
SSSSSSSSCSSCCSSCCSCCSSCCSCCSSCCSCCSSCCSCCSSCCSCCS
SCCSSS
>scTMHC2
(SEQ ID NO: 49)
MTRTEIIRELERSLRLQLVLAIFLMALLIVLLWLQQNGSSNNNVNYLLI
VILVLVLVIVALAVTQKYLVEQLKRQADPTDDSRTEIIRELERSLRLQL
VLAIFLMALLIVLLWLQQNGSSNNNVNYLLIVILVLVLVIVALAVTQKY
LVEQLKRQD
SSCSSCCSSCCSCCSSCSSCCSSCCSCCSSCCSCCSSSSCCSSCCSCCS
SCSSCCSSCCSCCSSCSSCCSSCSSSCSSSCSSCCSCCSSCCSCCSSCC
SCCSSCCSCCSSCSSCCSSSSCCSSCCSCCSSCCSCCSSCCSCCSSCSS
CCSSCCSSS

In another embodiment of any of the polypeptides disclosed herein, the polypeptide further comprising one or more bioactive polypeptides. As used herein, a “bioactive polypeptide” is any polypeptide that has an activity that adds functionality to the polypeptides of the disclosure. In non-limiting embodiments, such bioactive polypeptides may comprise polypeptide antigens, polypeptide therapeutics, detectable markers, scaffold proteins, etc. In various embodiments, the one or more bioactive polypeptide is present in the X1, X2, X3, or X4 domain, or wherein the one or more bioactive polypeptide is fused to the N-terminus or C-terminus of the polypeptide.

As used throughout the present application, the term “polypeptide” is used in its broadest sense to refer to a sequence of subunit amino acids. The polypeptides described herein may be chemically synthesized or recombinantly expressed. The polypeptides may be linked to other compounds to promote an increased half-life in vivo, such as by PEGylation, HESylation, PASylation, glycosylation, or may be produced as an Fc-fusion or in deimmunized variants. Such linkage can be covalent or non-covalent as is understood by those of skill in the art.

In another aspect the disclosure provides nucleic acids encoding the polypeptide of any embodiment or combination of embodiments of the disclosure. The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, eodysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the nucleic acids or expression vectors (i.e.: episomal or chromosomally integrated) disclosed herein, wherein the host cells can be either prokaryotic or eukaryotic. The cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated transfection.

The polypeptides, nucleic acids, expression vectors, and host cells of the disclosure may be used for any suitable purpose, as described in detail herein. In various non-limiting embodiments, the purpose may include displaying an antigen on a membrane (for example, for use as a vaccine); as a membrane localization marker; and/or as a stable scaffold to stabilize a target protein. In one embodiment the use comprises

a. providing one or more cells comprising the polypeptide, wherein the transmembrane domains of the polypeptides span the cellular membrane of the cell, and wherein the one or more polypeptides comprise extracellulaly presented bioactive polypeptide (as described herein);

b. admixing a sample with the one or more cells sufficient to allow binding of one or more agents in the sample (including but not limited to proteins and antibodies) with the extracellularly presented bioactive polypeptide; and

c. detecting the binding of the one or more agents with the extracellularly presented bioactive polypeptide.

### Examples

A major challenge for membrane protein design stems from the similarity of the membrane environment to protein hydrophobic cores. In the design of soluble proteins, the secondary structure and overall topology can be specified by the pattern of hydrophobic and hydrophilic residues, with the former inside the protein and the latter outside facing solvent. This core design principle cannot be used for membrane proteins, as the apolar environment of the hydrocarbon core of the lipid bilayer requires that outward facing residues in the membrane also be nonpolar.

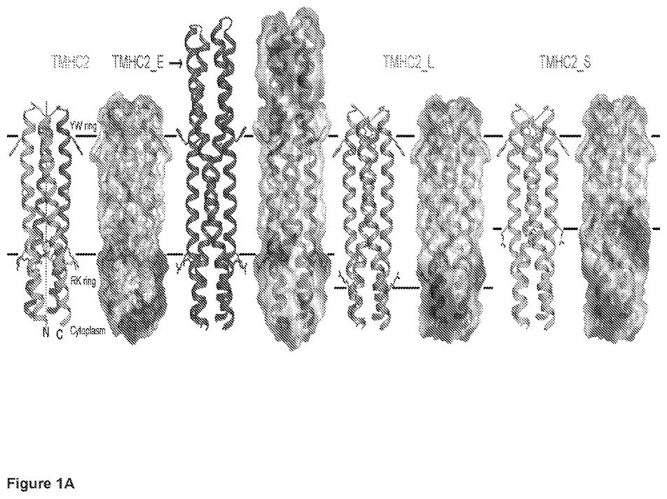

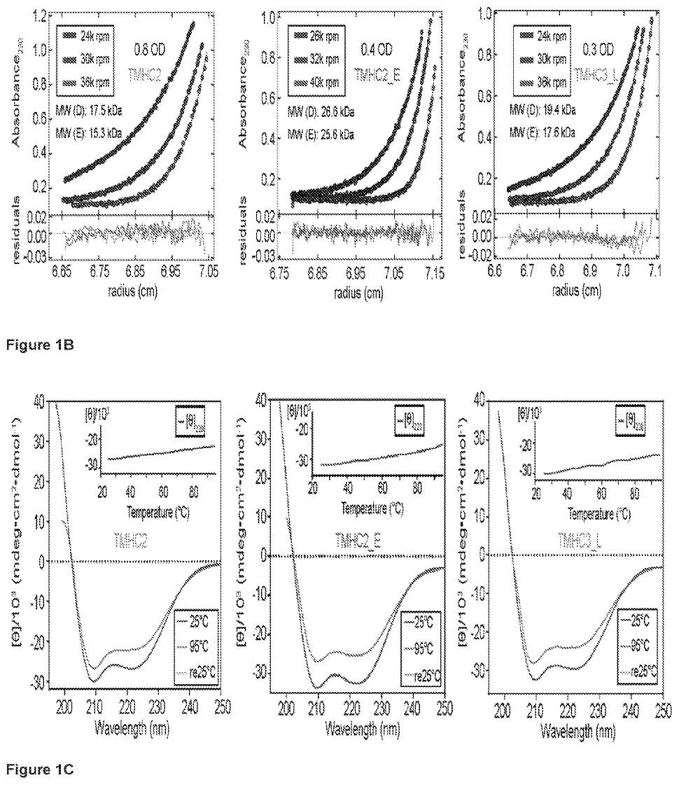

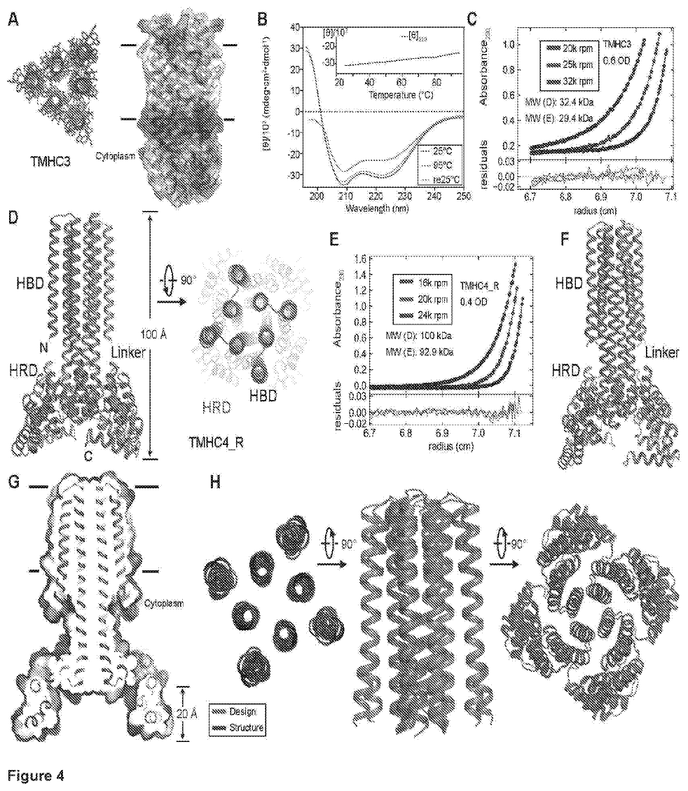

We first explored the design of helical transmembrane proteins with four transmembrane segments (TMs)—dimers of 76-to-104 residue hairpins or a single chain dimer of 156 residues—with hydrophobic spanning regions ranging from 21 to 35 Å (FIG. 1A and FIG. 2A), repurposing the Ser and Gln containing hydrogen bond networks in a designed soluble four-helix dimer with C2 symmetry (2L4HC2_23, (Protein Data Bank (PDB) ID: 5J0K)) to provide structural specificity. Four-helix bundles of different lengths with backbone geometries capable of hosting these networks were produced using parametric generating equations, residues comprising the hydrogen bond networks and neighboring packing residues were introduced, and the remainder of the sequence was optimized using Rosetta™ Monte Carlo design calculations to obtain low energy sequences. Connecting loops between the helices were built using Rosetta™. To specify the orientation of the designs in the membrane when expressed in cells, at the designed lipid-water boundary on the extracellular/periplasmic side we incorporated a ring of amphipathic aromatic residues and at the lipid-water boundary on the cytoplasmic side, a ring of positively charged residues (FIG. 1A and FIG. 2A). Between these two rings, the surface residues are exposed to the hydrophobic membrane environment; these positions in Rosetta™ sequence design calculations were restricted to hydrophobic amino acids [see supplementary materials]. Consistent with the design, TMHMM predicts that the dimer designs contain 2 TMs and the single chain design (scTMHC2), 4 TMs (FIG. 5). On average, for each residue ˜68% of the sidechain surface area is buried in the designs, which could provide substantial van der Waals stabilization.

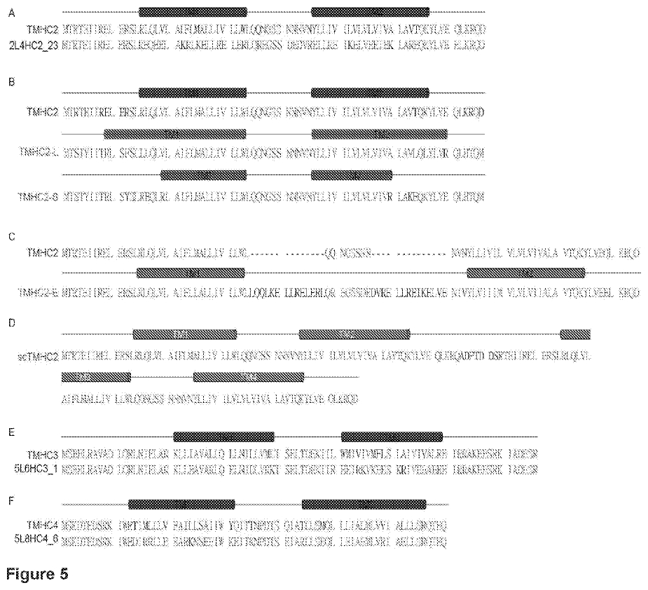

Synthetic genes encoding the designs were obtained and the proteins expressed in E. coli and mammalian cells using membrane protein expression vectors. The dimer design with the shortest hydrophobic span (15 residues, TMHC2_S) was poorly behaved in both E. coli and mammalian cells, but the dimer designs with longer spans TMHC2, TMHC2_E and TMHC2_L localized to the cell membrane when expressed in HEK293T cells (data not shown) and in E. coli. The designed proteins were purified by extracting the E. coli membrane fraction with detergent, followed by nickel-NTA chromatography and size exclusion chromatography (SEC) with a yield of ˜2 mg/L (FIG. 6A-B). The designed proteins TMHC2, TMHC2_E and TMHC2_L eluted as single peaks in SEC, and in analytical ultracentrifugation (AUC) experiments in detergent solution, the proteins sedimented as dimers consistent with the design models (exemplary data shown in FIG. 1B). For the single chain scTMHC2 the major species in SEC was the monomer with a small side peak that was readily removed by purification (FIG. 6B). Circular dichroism (CD) measurements showed that the designs were alpha helical and highly thermal stable—the CD spectra at 95° were similar to those at 25° (FIG. 1C and FIG. 2B). TOXCAT™-β-lactamase (TPL) assays, which couple E. coli survival to oligomerization and proper orientation of fused antibiotic resistance markers on the N and C termini, suggest that the N- and C-termini of TMHC2 are in the cytoplasm as in the design models (data not shown).

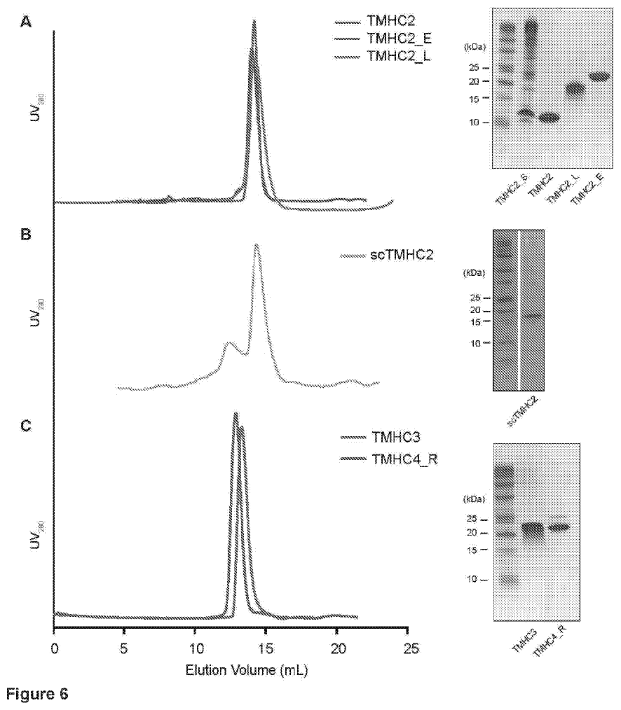

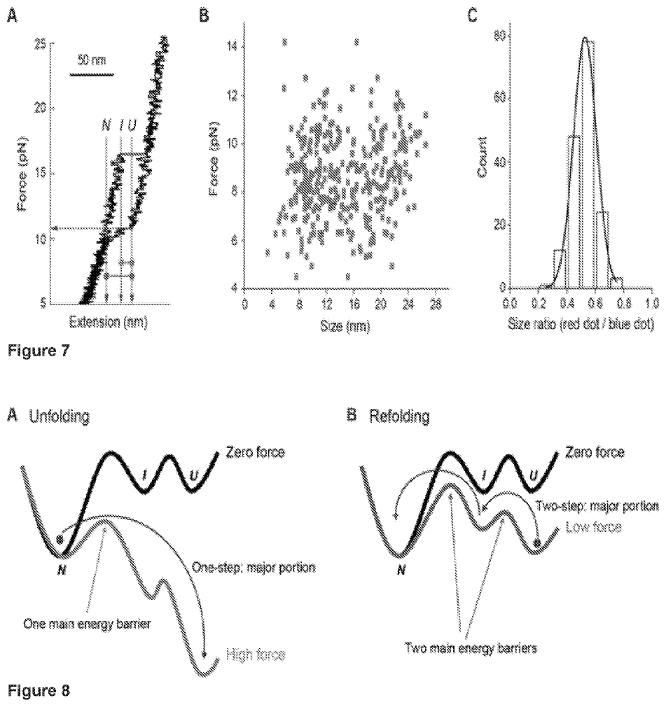

We more quantitatively characterized the folding stability of scTMHC2 using single-molecule forced unfolding experiments (FIG. 2). The designed protein reconstituted in a bicelle was covalently attached to a magnetic bead and a glass surface through its N- and C-termini (FIG. 2A). The distance between the bead and the surface was determined as a function of the applied mechanical tension. In unfolding experiments with the force slowly increasing (˜0.5 pN/s), unfolding transitions were observed at ˜18 pN and, upon force de-ramping, refolding transitions were observed at ˜9 pN (80.1% of the recorded unfolding traces had one step unfolding transitions and 84.6% of the refolding transitions had two steps; FIG. 2C). Consistent with the internal symmetry of the single-chain homodimer design (FIG. 2A), the two refolding step sizes were very similar (FIG. 7). This unfolding and refolding asymmetry is consistent with a three-state free energy landscape: a native dimer state (N), an intermediate state containing only one hairpin (I), and an unfolded state (U) (FIG. 8). During unfolding at high force, only the barrier between the native and intermediate states is observed, while at the lower forces where refolding occurs, both energy barriers become prominent (FIG. 8). The transition rates between the folded, intermediate and unfolded states were determined using the Bell model, yielding the relative free energies of the states and the associated barrier heights (FIG. 2D). The overall thermodynamic stability of scTMHC2 is 7.8(±0.9) kcal/mol—on a per transmembrane helix basis, more stable than the naturally occurring helical membrane proteins studied thus far (folding free energy per helix for scTMHC2 is 2.0(±0.2) kcal/(mol·helix) compared to 0.7-0.9 kcal/(mol·helix) for GlpG (14, 17) and 1.6-1.8 kcal/(mol·helix) for bacteriorhodopsin).

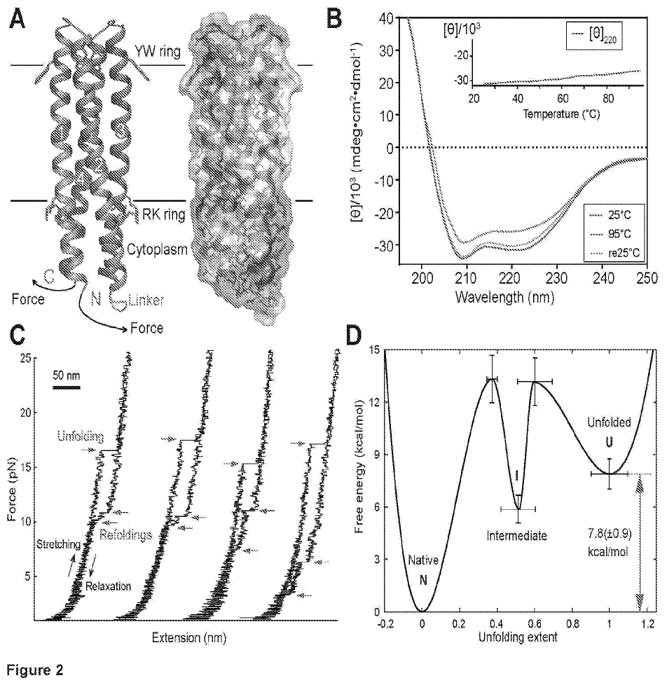

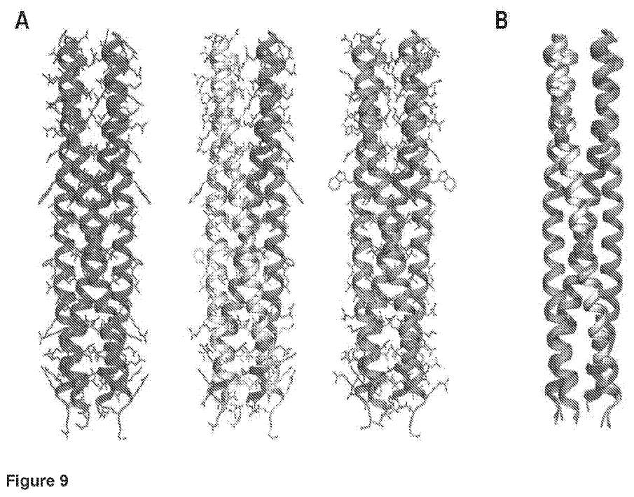

We carried out crystal screens in different detergents for each of the designs, and obtained crystals of the design with the most extensive cytoplasmic region, TMHC2_E, in n-nonyl-β-D-glucopyranoside (NG). The crystals diffracted to 2.95 Å resolution, and we solved the structure by molecular replacement with the design model. As anticipated, the extended soluble region mediates the crystal lattice packing; there are large solvent channels around the designed TMs likely due to the surrounding disordered detergent molecules (FIG. 3A). Each asymmetric unit contains four helical hairpins, two are paired in a dimer while the other two form two C2 dimers through crystallographic symmetry with two monomers in adjacent asymmetric units; the C2 axis in the design is perfectly aligned with the crystallographic two fold (FIG. 3B). The conformations of the dimers in the three biological units are nearly identical with very small differences due to crystal packing (Cα root-mean-square deviations (RMSDs): 0.60-0.84 Å) (FIG. 9). Both the overall structure and the core sidechain packing are almost identical in the crystal structure and the design model with a Cα RMSD of 0.7 Å over the core residues (FIG. 3C). Two of the three buried hydrogen bonding residues within the membrane have conformations that almost exactly match the design model (S13 and Q93), but 017 adopts a different rotamer with the side-chain nitrogen donating a hydrogen bond to the main-chain carbonyl oxygen (FIG. 3D).

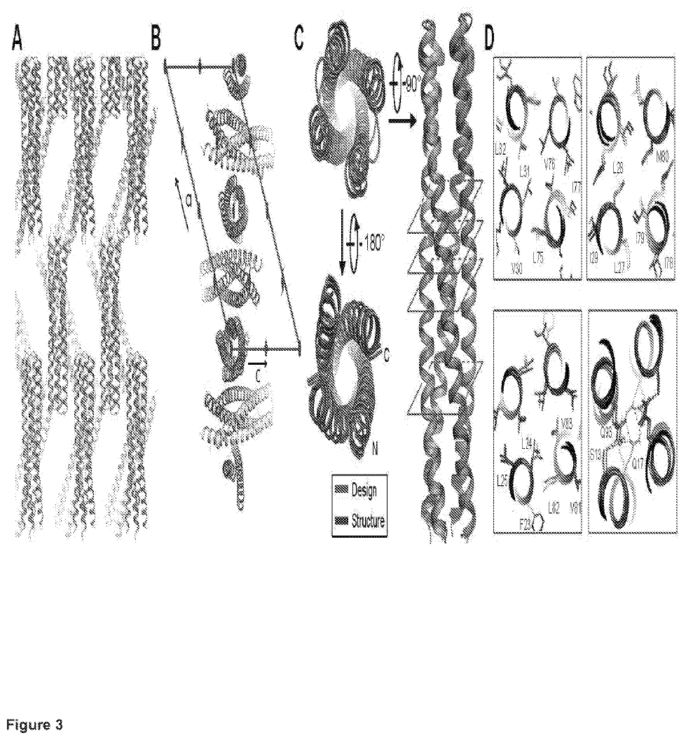

We used a similar approach to design a transmembrane trimer with six membrane spanning helices (TMHC3) based on the 5L6HC3_1 scaffold (PDB ID: 5IZS). Guided by the results with the C2 designs, we chose a hydrophobic span of ˜30 Å (20 residues) (FIG. 4A). The design was expressed in E. coli and purified to homogeneity, eluting on a gel filtration column as a single homogeneous species (FIG. 5C). CD measurements showed that TMHC3 was highly thermostable with the alpha helical structure preserved at 95° (FIG. 4B). AUC experiments showed that TMHC3 is a trimer in detergent solution consistent with the design (FIG. 4C).

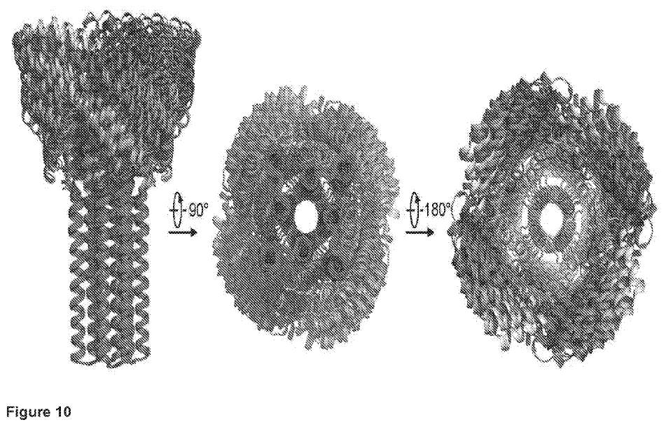

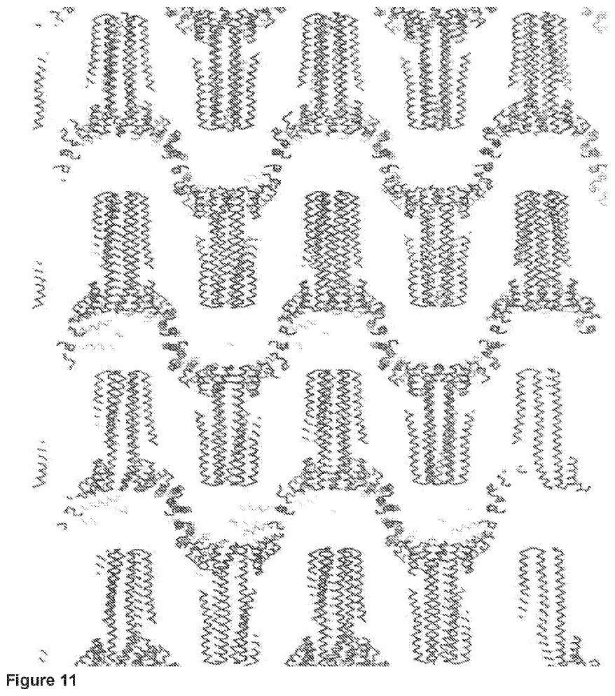

To explore our capability to design membrane proteins with more complex topologies, we designed a C4 tetramer with a two ring helical bundle membrane spanning region composed of 8 TMs and an extended bowl shaped cytoplasmic domain formed by repeating structures emanating away from the symmetry axis (FIG. 4D). The design has an overall rocket shape with a height of ˜100 Å and can be divided into three regions: the helical bundle domain (HBD), the helical repeat domain (HRD), and the helical linker between the two. The central HBD was derived from the soluble design 5L8HC4_6 and the bowl from a designed helical repeat protein homo-oligomer (tpr1C4_2). Helical linkers were built using RosettaRemodel™—a 9-residue junction was found to yield the correct helical register (FIG. 10). Following Rosetta™ sequence design calculations, a gene encoding the lowest energy design, TMHC4_R, was synthesized. The protein was expressed in E. coli and purified using nickel affinity and gel filtration chromatography; the final yield was ˜3 mg/L and the purified protein chromatographed as a monodisperse peak in SEC (FIG. 6C). CD experiments showed that the design was alpha-helical and thermostable up to 95° (data not shown). AUC measurements showed that TMHC4_R is a tetramer in detergent solution, consistent with the design model (FIG. 4E). After a systematic effort to screen detergents for crystallization, we obtained crystals in a combination of n-Decyl-β-D-Maltopyranoside (DM) and NG in the P4 space group that diffracted to 3.9 Å resolution. We solved the crystal structure by molecular replacement using the design model (R_{work}/R_{free}=0.29/0.32 with unambiguous electron density) (Table 1). The crystal lattice packing is primarily between the extended cytoplasmic domains; there may be minor detergent-mediated interactions between the transmembrane and helical repeat (HR) domains as well (FIG. 11).

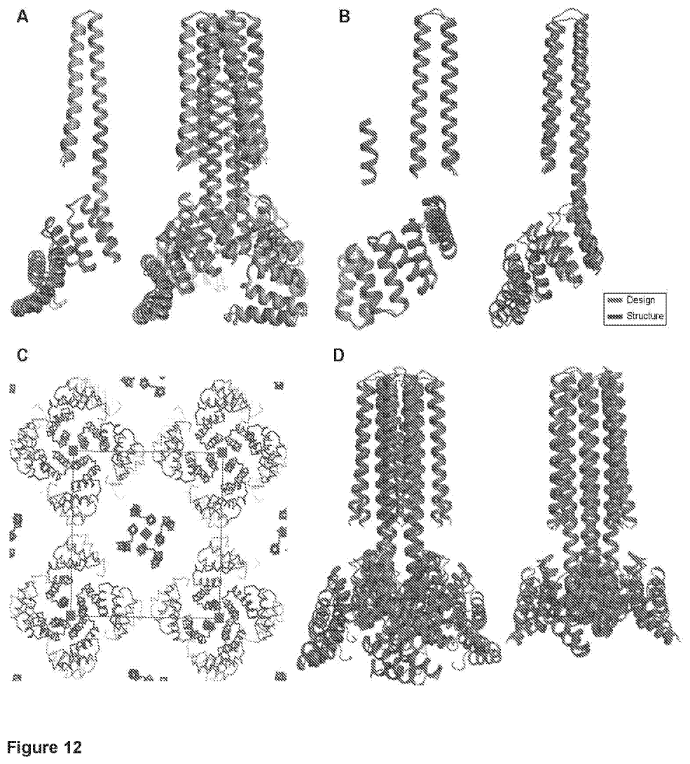

Although the resolution is insufficient for evaluating the details of the side-chain packing, it does allow backbone-level comparisons. There are four TMHC4_R monomers in one asymmetric unit, with nearly identical structures (Cα RMSDs between 0.2 and 0.6 Å) (FIG. 12A). The Cα RMSDs between the structure and design model are 1.2-1.8 Å for the monomer transmembrane helices, 0.3-0.4 Å for the linkers, 1.1-1.5 Å for the HR domains, and 3.3-3.6 Å for the overall structure (FIG. 12B). As in the case of the C2 design, the C4 symmetry axis of the design coincides with the crystallographic axes of the crystal lattice (FIG. 12C). The four tetramer structures on the crystal C4 axes have overall structures very similar to each other and to the design model (FIG. 4F-G, and FIG. S12A); the tetrameric transmembrane domain, HR domain, and overall tetramer structure have Cα RMSDs to the design model of 1.3-1.5 Å, 3.3-3.8 Å and 3.3-3.8 Å, respectively (FIG. 4H and FIG. 12D, left panel). The deviation in the HR domain may result from crystal packing interactions between the termini; the Cα RMSDs over the first 162 residues are 2.2-2.3 Å (FIG. 12D, right panel). The main deviation from the design model is a tilting of the outer helices of transmembrane hairpins from the axis by ˜10° (FIG. 4F-G).

The agreement between the crystal structures of TMHC2_E and TMHC4_R with the design models demonstrates that transmembrane homo-oligomers containing multiple membrane spanning regions and extensive extracellular domains were accurately designed. Our general approach of first designing and characterizing hydrogen bond network containing soluble versions of the desired transmembrane structures, and then converting to integral membrane proteins by redesigning the membrane exposed residues, was shown to be quite robust. Single-molecule forced unfolding and thermal denaturation experiments show that the designed proteins are highly stable. The designed proteins bury more surface area than typical soluble proteins, thereby maximizing van der Waals packing contributions. The range of the design features-variable transmembrane and extracellular helix lengths and twists, extensive soluble domains and diverse oligomeric states-demonstrate the ability to design transmembrane proteins with multiple membrane spanning regions and extra membrane domains that play important roles in ligand/substrate recognition and structure stabilization as in the ATP binding cassette (ABC) transporters, ion channels, ryanodine receptor and gamma-secretase.

### Materials and Methods

### Computational Modeling

### Transmembrane Region Design

### Orientation, RK Ring and YW Ring

The orientations of natural transmembrane proteins across the membrane follow the positive-inside rule—that is, the side which is more positively charged, probably containing more Arg and Lys residues, would be in the cytoplasm. For transmembrane proteins with even numbers of TMs, the N- and C-termini are preferred to localize in the cytoplasmic side. The N- and C-termini of the designs made in this study are designed facing the cytoplasmic side, through adding a ring of Arg and Lys residues, named “RK ring”, close to the N- and C-termini end of the helical bundle and designing the Arg and Lys to other polar residues on the other end. Only the changes that would not clash are accepted during the design. Amphipathic aromatic residues (i.e., Trp and Tyr) prefer to locate at lipid-water boundary, forming a “YW ring”. Trp and Tyr residues may interact with the lipid headgroups and water molecules in the boundary region and also pack with the lipid aliphatic chains, locking the transmembrane protein with the right register in membrane. The YW ring is designed on the other end of the RK ring, without steric clash.

### Definition of Hydrophobic Transmembrane Span

The hydrophobic transmembrane span could be defined as the region between the YW and KR rings. As all the designs have central symmetry, the central symmetry axis of designs may be perpendicular to the membrane plane; otherwise more hydrophobic and hydrophilic residues will be exposed to water solvent and buried in lipid membrane, respectively, which is energetically unfavorable. The center symmetry axis is aligned to the z axis, thus, the length of hydrophobic transmembrane region could be expressed as the distance between the mean z-coordinate values of the Cα atoms of YW and KR rings. We tested the lengths ranging from 21 to 35 Å.

### Rosetta™ Calculation

RosettaMP™ uses a “span” object to store the start and end residue numbers of a single transmembrane span. An updated score function, which is derived from the original RosettaMembrane™ score functions, is implemented in RosettaMP™. RosettaMP™ uses the membrane position to score per-residue and residue pair interactions within the hydrophobic layers. The restructured membrane score function was verified using continuous regression testing and showed good scientific integrity.

Between the YW and KR rings, diverse hydrophobic residues were designed to replace all the polar residues those with polar atoms not involved in any hydrogen bond network, based on amino acid propensity in the membrane. The diversity could be achieved by application of an amino acid composition based energy term (“aa_composition”) in the design energy function that penalized sequences possessing too many similar nonpolar amino acids. Sometimes Phe could be designed at positions roughly in the middle of the TM region, again, without causing any clash.

### Helices Extension

We used the Crick coiled-coil parameters of 2L4HC2_23 but with lengths up to 14 more residues per helix, which form two additional full helical turns. The same hydrogen bond networks were introduced by specifying the residues at corresponding positions, and the remainder of the sequence was designed using Rosetta™ Monte Carlo calculations. The helices were connected into a single chain by adding loops using look-ups to a structural database and Rosetta™ design. Briefly, we generated an exhaustive database of loop backbones, spanning two helical regions with five or less residues. Candidate loops were identified via the alignment of the terminal residues of the elongated helical bundle to the database. Candidates within 0.35 Å root-mean-square deviation (RMSD) were then designed using Rosetta™ Monte Carlo design calculations and the lowest-scoring candidate is selected as the final loop design.

### Junction Design

RosettaRemodel™ protocol was used to find the α-helical junction that can connect the helical bundle domain and helical repeat protein domain of TMHC4_R. We set up sampling runs for the junction lengths from 0 to 10 residues under four-fold symmetry. Distance constraints between the subunits of the tetrameric helical repeat protein and total energy are used for selection of the optimal helix length, which was found to be 9—other lengths either changed the helical register shifts or caused clashes. The models chosen from the fragment sampling stage for final sequence refinement are subjected to Rosetta Monte Carlo design calculations based on layer design protocol (30) to obtain low energy sequences, the sequences are converged quickly and the design with the lowest score are selected for experimental test.

### Structural Figures

All structural images for figures were generated using PyMOL™.

### Experimental Materials and Methods

### Reagents

Chemicals used were of the highest grade commercially available and were purchased from Sigma-Aldrich (St. Louis, Mo., USA), Invitrogen (Carlsbad, Calif., USA), or Qiagen (Hilden, Germany). Detergents were from Anatrace (Maumee, Ohio, USA) and crystallization reagents were from Hampton (Aliso Viejo, Calif., USA).

### Cloning and Expression

Synthetic genes were obtained from IDT (Coralville, Iowa, USA), Genscript Inc. (Piscataway, N.J., USA) and Gen9 Inc. (Cambridge, Mass., USA) and either delivered in pET29b expression vector or as linear dsDNA, and sub-cloned into pET-29b in-house via NdeI/XhoI restriction sites. The genes were designed without a stop codon, which allows expression of the protein with a C-terminal hexa-histidine tag. TMHC2 is cloned into pET-28b via NdeI/XhoI restriction sites, and with a N-terminal hexa-histidine tag followed by a thrombin cutting site. The assembled plasmids were transformed into chemically competent E. coli BL21(DE3)pLysS cells (Invitrogen). Gene expression was facilitated by growing pre-cultures in Luria-Bertani (LB) medium with a final concentration of 50 μg/ml kanamycin overnight at 37° C. 10 ml pre-cultures were used to inoculate 1 L of LB medium, again containing 50 μg/ml kanamycin for plasmid selection. The cultures were grown at 37° C. until an OD600 of 0.8-1.0 was reached and expression was induced by addition of isopropyl thio-β-D-galactoside (IPTG) to a final concentration of 0.2 mM. Protein was expressed at 18° C. overnight and cells were harvested by centrifugation.

### Cell Lysis and Purification

Cells were resuspended and homogenized in lysis buffer containing 25 mM Tris-HCl pH 8.0 and 150 mM NaCl. After further disruption with a French press, cell debris was removed by low-speed centrifugation for 10 min. The supernatant was collected and ultracentrifuged for 1 h at 150,000 g. The membrane fraction was collected and homogenized with buffer containing 25 mM Tris-HCl pH 8.0 and 150 mM NaCl. n-Decyl-A-D-Maltopyranoside (DM: Anatrace) was added to the membrane suspension to a final concentration of 1.5% (w/v) and then incubated for 2 h at 4° C. After another ultracentrifugation step at 150,000 g for 30 min, the supernatant was collected and loaded on Ni²⁺-nitrilotriacetate affinity resin (Ni-NTA; Qiagen), followed by a wash with 25 mM Tris-HCl pH 8.0, 150 mM NaCl, 30 mM imidazole and 0.2% DM. Proteins were eluted with buffer containing 25 mM Tris-HCl pH 8.0, 150 mM NaCl, 30 mM imidazole and 0.2% DM. After concentration to 10-15 mg ml⁻¹, proteins were further purified by gel filtration (Superdex-200 10/30; GE Healthcare). The buffer for gel filtration contained 25 mM Tris-HCl pH 8.0, 150 mM NaCl and various detergents. The purified proteins are separated on 16.5% Mini-PROTEAN® Tris-Tricine Gel (Bio-Rad) and visualized by Coomassie Blue staining. For TMHC2, the hexa-histidine tag is removed by cleavage of thrombin. After full cleavage, the reaction is stopped by addition of phenylmethanesulfonyl fluoride (PMSF), followed by another round of gel filtration purification. DM buffer is used for general purpose. For AUC experiments, the proteins were buffer exchanged in 20 mM sodium phosphate, pH 7.0, containing 200 mM NaCl supplemented with 0.5% Pentaethylene Glycol Monooctyl Ether (C8E5). For crystallization, different detergents are screened on gel filtration. The peak fractions were collected, concentrated to 10-15 mg ml⁻¹, aliquoted and flash frozen by liquid nitrogen.

### Crystallization

The hanging-drop vapour-diffusion method was performed at 20° C. during crystallization. For TMHC2_E, crystals belonging to the space group C2 were obtained with protein purified in the presence of 0.2% n-nonyl-A-D-glucopyranoside (f-NG; Anatrace). The crystallization buffer was 0.05 M magnesium acetate tetrahydrate, 0.05 M sodium acetate 5.5 and 24% v/v polyethylene glycol (PEG) 400. Rod cluster-shaped crystals appeared in 2-3 days and typically grew to full size in about 1 week. Single crystals could be obtained from one branch of the rod cluster. Crystals were dehydrated by exposing the drops to air for 5 min. For TMHC4_R, crystals in P4 space group were obtained in a detergents mixture of 0.2% β-NG and 0.1% DM. The crystallization buffer was 30% v/v PEG 400, 100 mM 3-(N-morpholino)propanesulfonic acid (MOPS) pH 7.0, 100 mM NaCl. 10 mM N,N-Dimethyldecylamine-N-oxide (DDAO) was identified in detergent additive screen, which would improve the crystal quality. Plate-shaped crystals appeared in 1 week and typically grew to full size in about 4 weeks.

### Data Collection and Structure Determination

Crystal diffraction data for TMHC2_E and TMHC4_R, were collected at ALS beamline BL8.2.1 and BL5.0.1, respectively, and processed with the package HKL-2000 (32) with routine procedures. The scaled data were then used for structural determination and refinement. Further processing was carried out with programs from the CCP4 suites (33). Data collection statistics are summarized in Supplementary Table 1. For TMHC2_E and TMHC4_R, the best diffraction reached 2.95 Å and 3.9 Å, respectively.

### Structure Determination of TMHC2_E

From the data, the apparent space group was I212121, and an MR solution was found by Phaser™ with TFZ=9.7, but refinement was unable to improve the structure. We then tried molecular replacement using Rosetta™ ab initio models and in lower symmetry groups. In doing so, we found a solution in C2 with four copies in the asymmetric unit: in two copies the designed dimer was part of the crystal symmetry, and the other two copies formed a dimer. Using Rosetta™-Phenix refinement (35), the system refined to R/R_{free}=0.258/0.276.

Structure Determination of TMHC4_R

Using the design model as well as ˜25 models perturbed with RosettaCM™, we were unable to find a solution in the apparent space group, P4212. After trying molecular replacement with lower symmetry, one of the perturbed models was able to place 4 copies in P4 (two pairs each related by tNCS). The original design model was inappropriate for MR as the angle between the transmembrane helices and repeat protein was different in the crystal lattice, however, several of the perturbed models accurately modeled this flexing, giving TFZ values of ˜11 once all four copies were placed. This solution in P4 was then straightforward to refine in Phenix-Rosetta, giving a final R/R_{free} of 0.291/0.322.

### Circular Dichroism (CD) Measurements

CD wavelength scan measurements were made on an AVIV CD spectrometer model 420. Protein concentrations ranged from 0.1-0.2 mg/ml in PBS (pH 7.4) buffer plus 0.2% DM. Wavelength scan spectra from 260 to 190 nm were recorded in triplets and averaged. The scanning increment for full wavelength scans was 1 nm. Temperature melts were conducted in 2° C. steps (heating rate of 2° C./min) and recorded by following the absorption signal at a wavelength of 220 nm. Three sets of wavelength scan spectra were recorded at 25° C., 95° C. and after cooled down to 25° C.

### TOXCAT™-β-Lactamase (TPL) Assays

TPL assay is a genetic screen based on insertion of membrane-spanning segment to the N-terminus ToxR and C-terminus β-lactamase. ToxR is an oligomerization-dependent transcriptional activator, which could activate a chloramphenicol-resistance gene in this system. Bacterial survival on ampicillin monitors periplasmic localization of the C-terminus, and survival on chloramphenicol correlates with self-association of the membrane span and cytoplasmic localization of the N-terminus. The genes encoding TM designs were cloned into p-Mal vector using XhoI and SpeI restriction sites, and selected by spectinomycin. The TMs of the human erythrocyte sialoglycoprotein Glycophorin A (GpA) is used as a positive control. The resulting plasmids were transformed into E. coli XL-1 blue (Agilent), plated on agar plates containing 50 μg/ml spectinomycin, and used to inoculate 10 ml of Luria Broth medium (LB) with 50 μg/ml spectinomycin and grown in a shaker at 200 rpm and 37° C. overnight. The cultures were then inoculated into fresh medium, and until the density reached OD₆₀₀=1. 1 μl of the resulting cultures were plated at different dilutions on large 12-cm petri dishes containing spectinomycin, ampicillin alone or chloramphenicol.

### Cell Localization

Synthetic genes (codon optimized for human expression) were obtained from IDT and subcloned into pCAGGS vector via NheI and XhoI along with a fluorescent c-terminal protein tag (i.e., mTagBFP, eGFP, or mCherry). HEK293T cells were transiently transfected using TransIT™-293T transfection reagent (Mirus Bio) along with constructs encoding the synthetic transmembrane proteins fused to a fluorescent tag. After 12-24 hours, cells were detached by incubation in PBS+2 mM EDTA (Thermo Fisher Scientific, Sigma-Aldrich) for 4 minutes at room temperature. Cells were then transferred into Opti™-MEM+10% FBS (Thermo Fisher Scientific), seeded in 8 chambered coverglass wells (In Vitro Scientific) pre-coated with 1 mg/ml fibronectin (Thermo Fisher Scientific), and incubated for >4 hours to overnight at 37° C. Wells were imaged on a spinning-disk confocal microscope (Nikon) at 60×. A line-scan through a region of the plasma membrane was performed using FIJI to determine if the protein of interest localized to the membrane.

### Analytical Ultracentrifugation

Analytical ultracentrifugation (sedimentation velocity and sedimentation equilibrium) experiments were carried out using a Beckman XL-I analytical ultracentrifuge (Beckman Coulter) equipped with an eight-cell An-50 Ti rotor. The proteins were run in 20 mM sodium phosphate, pH 7.0, containing 200 mM NaCl supplemented with 0.5% C8E5, no density matching was necessary and the solvent density was calculated as 1.0075 g mL⁻¹. The partial specific volume of the protein was calculated by the program Sednterp™ (37). For sedimentation velocity, absorbance at 230 nm versus radial location was recorded during centrifugation at 50,000 rpm at 20° C. For sedimentation equilibrium, data were collected by UV detector at 20° C. for at least two protein concentrations at three rotor speeds. The data of sedimentation velocity and sedimentation equilibrium were analyzed using Sedfit™ and Sedphat™.

TABLE 1
Statistics of data collection and refinement

| Data | TMHC2_E | | TMHC4_R | |
|---|---|---|---|---|
| integration Package | HKL2000 | | HKL2000 | |
| Space Group | C2 | | P4 | |
| Content per ASU | 4 monomers | | 4 monomers | |
| Unit Cell (Å) | 103.5, 121.6, 52.0 | | 80.2, 80.2, 251.6 | |
| Unit Cell (°) | 90, 119.9, 90 | | 90, 90, 90 | |
| Resolution (Å) | 50~2.95 | (3.03~2.95) | 50~3.9 | (4.01~3.90) |
| Outer shell (Å) | | | | |
| R_{merge} | 0.097 | (0.635) | 0.133 | (2.065) |
| I/sigma | 9.6 | (1.4) | 18.2 | (1.0) |
| CC_{1/2} | 0.688 | | 0.398 | |
| Completeness (%) | 92.5 | (60.7) | 99.6 | (97.4) |
| Number of unique | 10,899 | | 14,545 | |
| reflections | | | | |
| Redundancy | 3.5 | (2.6) | 12.0 | (8.7) |

| R_{work}/R_{free} | 0.258/0.276 | 0.291/0.322 |
|---|---|---|
| No. atoms | | |
| Overall | 6508 | 6764 |
| Protein | 6508 | 6764 |
| Water | 0 | 0 |
| Other entities | 0 | 0 |
| Average B value (Å²) | | |
| Protein | 84.8 | 172.5 |
| Water | N/A | N/A |
| Other entities | N/A | N/A |
| R.m.s. deviations | | |
| Bonds (Å) | 0.011 | 0.021 |
| Angle (°) | 1.257 | 1.558 |
| Ramachandran plot | | |
| statistics (%) | | |
| Most favourable | 100 | 99.4 |
| Additionally allowed | 0.0 | 5.9 |
| Generously allowed | 0.0 | 0.0 |
| Disallowed | 0.0 | 0.0 |

Every diffraction dataset was collected from a single crystal. Values in parentheses are for the highest resolution shell. R_{merge}=ΣₕΣᵢ|I_{h,i}−Iₕ|/ΣₕΣᵢI_{h,i}, where Iₕ is the mean intensity of the i observations of symmetry related reflections of h. R=Σ|F_{obs}−F_{calc}|/ΣF_{obs}, where F_{calc} is the calculated protein structure factor from the atomic model (R_{free} was calculated with 5% of the reflections selected).

1. A non-naturally occurring polypeptide comprising the general formula X1-TM1-X2-TM2-X3, wherein
X1 is an optional first peptide domain
TM1 is a first transmembrane peptide of between 15 and 35 amino acids in length and capable of spanning a biological membrane, wherein (a) the first residue of TM1 is R or K; (b) the last residue of TM1 is W, Y, or L; and (c) at least 60%, 65%, 70%, 75%, 80%, 85%, 90%, or more of the internal residues are hydrophobic;
X2 comprises a first connecting peptide;
TM2 is a second transmembrane peptide of between 15 and 35 amino acids in length and capable of spanning a biological membrane, wherein (a) the first residue of TM2 is W, T, Q, or Y; (b) the last residue of TM2 is R or K; and (c) at least 60%, 65%, 70%, 75%, 80%, 85%, 90%, or more of the internal residues are hydrophobic; and
X3 is an optional second peptide domain;
wherein TM1 includes at least a first interior polar amino acid residue that is capable of forming a hydrogen bond with a first interior polar amino acid residue present in TM2.

2. The polypeptide of claim 1, wherein TM1 and TM2 each include at least two interior polar amino acid residues capable of hydrogen bonding with interior amino acids of the other TM domain.

3.-5. (canceled)

6. The polypeptide of claim 1, wherein TM1 comprises the internal amino acid sequence LAXXL(M/L)XLLXXLL (SEQ ID NO: 1), wherein “X” is any hydrophobic amino acid, or wherein TM1 comprises the internal amino acid sequence LAIFL(M/L)ALLIVLL (SEO ID NO: 2).

7. (canceled)

8. The polypeptide of claim 1, wherein TM1 comprises the amino acid sequence selected from the group consisting of SEQ ID NOS:3-13 wherein “X” is any hydrophobic amino acid:
TMHC2 and cTMHC2
(SEQ ID NO: 3)
(R/K)XQXXLAXXLMXLLXXLL(W/Y/L)
TMHC2_L
(SEQ ID NO: 4)
(R/K)LSXSLXXQLXLAXXLMXLLXXLL(W/Y/L)
TMHC2_S
(SEQ ID NO: 5)
(R/K)LAXXLMXLLXXLL(W/Y/L)
TMHC2_E
(SEQ ID NO: 6)
(R/K)XQLXLAXXLLXLLXXLL(W/Y/L)
TMHC2_E_V1
(SEQ ID NO: 7)
(R/K)LSXSLXXQLXLAXXLLXLLXXLLW
TMHC2_E_V2
(SEQ ID NO: 8)
(R/K)LSXSLXXQLXLAXXLLXLLXXLLXLLX(Y/W/L),
TMHC2 and scTMHC2
(SEQ ID NO: 10)
RLQLVLAIFLAMLLIVLLW
TMHC2_L
(SEQ ID NO: 9)
RLSFSLLLQLVLAIFLMALLIVLLW
TMHC2_S
(SEQ ID NO: 14)
RLAIFLMALLIVLLW
TMHC2_E
(SEQ ID NO: 11)
RLQLVLAIFLLALLIVLLW
TMHC2_E_V1
(SEQ ID NO: 12)
RLSFSLLLQLVLAIFLLALLIVLLW
TMHC2_E_V2
(SEQ ID NO: 13)
RLSFSLLLQLVLAIFLLALLIVLLVLLIY

9. (canceled)

10. The polypeptide of claim 1, wherein TM2 comprises the amino acid sequence XL(L/V)XXI(L/M)XLVXXI(V/I)X (SEQ ID NO: 15), wherein X is any hydrophobic amino acid, or wherein TM2 comprises the amino acid sequence (Y/A)L(L/V)I(V/I)I(L/M)VLVLVI(V/I(A/R) (SEO ID NO: 16).

11. (canceled)

12. The polypeptide of claim 1, wherein TM2 comprises the amino acid sequence selected from the group consisting of SEQ ID NOS:17-29 wherein X is any hydrophobic amino acid, and Z is any polar amino acid:
TMHC2
(SEQ ID NO: 17)
(W/T/Q/Y)LLXXILXLVXXIVXLAXZQ(K/R)
TMHC2_L
(SEQ ID NO: 18)
(W/T/Q/Y)LLXXIXXLVXXIVXLAXXQXZLV(R/K)
TMHC2_S
(SEQ ID NO: 19)
(W/T/Q/Y)LLXXIXXLVXXIV(R/K)
TMHC2_E
(SEQ ID NO: 20)
(W/T/Q/Y)LVXXIMXLVXXIIXLAXZQ(K/R)
TMHC2_E_V1
(SEQ ID NO: 21)
(W/T/Q/Y)LVXXIMXLVXXIIXLAXXQMZXX(R/K)
TMHC2_E_V2
(SEQ ID NO: 22)
(W/T/Q/Y)LVXXIVXLVXXIMXLVXXIIXLAXXQMZLV(R/K)
scTMHC2
(SEQ ID NO: 23)
(W/T/Q/Y)LLXXIXXLVXXIVXLAXZQ(K/R);
TMHC2 and scTMHC2
(SEQ ID NO: 24)
YLLIVILVLVLVIVALAVTQK
TMHC2_L
(SEQ ID NO: 25)
YLLIVILVLVLVIVALAVLQLYLVR
TMHC2_S
(SEQ ID NO: 26)
YLLIVILVLVLVIVR
TMHC2_E
(SEQ ID NO: 27)
YLVIIIMVLVLVIIALAVTQK
TMHC2_E_V1
(SEQ ID NO: 28)
YLVIIIMVLVLVIIALAVLQMYLVR
TMHC2_E_V2
(SEQ ID NO: 29)
WLVIVIVALVIIIMVLVLVIIALAVLQMYLVR.

13. (canceled)

14. The polypeptide of claim 1, wherein TM1 and TM2 comprise a pair selected from the group consisting of:
(a) TM1 comprises the amino acid sequence (R/K)XQXXLAXXLMXLLXXLL(W/Y/L) (SEQ ID NO: 3) and TM2 comprises the amino acid sequence (W/T/Q/Y)LLXXILXLVXXIVXLAXZQ(K/RX TMHC2) (SEQ ID NO: 17);
(b) TM1 comprises the amino acid sequence (R/K)LSXSLXXQLXLAXXLMXLLXXLL(W/Y/L) (SEQ ID NO: 4) and TM2 comprises the amino acid sequence (W/T/Q/Y)LLXXIXXLVXXIVXLAXXQXZLV(R/K) (SEQ ID NO: 18) (TMHC2_L);
(c) TM1 comprises the amino acid sequence (R/K)LAXXLMXLLXXLL(W/Y/L) (SEQ ID NO: 5) and TM2 comprises the amino acid sequence (W/T/Q/Y)LLXXIXXLVXXIV(R/K) (SEQ ID NO: 19) (TMHC2_S);
(d) TM1 comprises the amino acid sequence (R/K)XQLXLAXXLLXLLXXLL(W/Y/L) (SEQ ID NO: 6) and TM2 comprises the amino acid sequence (W/T/Q/Y)LVXXIMXLVXXIIXLAXZQ(K/R) (SEQ ID NO: 20) (TMHC2_E);
(e) TM1 comprises the amino acid sequence (R/K)LSXSLXXQLXLAXXLLXLLXXLL(W/Y/L) (SEQ ID NO: 30) and TM2 comprises the amino acid sequence (W/T/Q/Y)LVXXIMXLVXXIIXLAXXQMZXX(R/K) (SEQ ID NO: 21) (TMHC2_E_V1);
(f) TM1 comprises the amino acid sequence (R/K)LSXSLXXQLXLAXXLLXLLXXLLXLLX(Y/W/L) (SEQ ID NO: 8) and TM2 comprises the amino acid sequence (W/T/Q/Y)LVXXIVXLVXXIMXLVXXIIXLAXXQMZLV(R/K) (SEQ ID NO: 22) (TMHC2_E_V2); and
(g) TM1 comprises the amino acid sequence (R/K)XQXXLAXXLMXLLXXLL(W/Y/L) (SEQ ID NO:3) and TM2 comprises the amino acid sequence (W/T/Q/Y)LLXXIXXLVXXIVXLAXZQ(K/R) (SEQ ID NO: 23);
wherein X is any hydrophobic amino acid and Z is any polar amino acid.

15. The polypeptide of claim 1, wherein TM1 and TM2 comprise a pair selected from the group consisting of:
(a) TM 1 comprises the amino acid sequence RLQLVLAIFLMALLIVLLW (SEQ ID NO: 10) and TM2 comprises the amino acid sequence YLLIVILVLVLVIVALAVTQK (SEQ ID NO: 24) (TMHC2);
(b) TM 1 comprises the amino acid sequence RLSFSLLLQLVLAIFLMALLIVLLW (SEQ ID NO: 9) and TM2 comprises the amino acid sequence YLLIVILVLVLVIVALAVLQLYLVR (SEQ ID NO: 25) (TMHC2_L);
(c) TM 1 comprises the amino acid sequence RLAIFLMALLIVLLW (SEQ ID NO: 14) and TM2 comprises the amino acid sequence YLLIVILVLVLVIVR (SEQ ID NO: 26) (TMHC2_S);
(d) TM 1 comprises the amino acid sequence RLQLVLAIFLLALLIVLLW (SEQ ID NO: 11) and TM2 comprises the amino acid sequence YLVIIIMVLVLVIIALAVTQK (SEQ ID NO: 27) (TMHC2_E);
(e) TM 1 comprises the amino acid sequence RLSFSLLLQLVLAIFLLALLIVLLW (SEQ ID NO: 12) and TM2 comprises the amino acid sequence YLVIIIMVLVLVIIALAVLQMYLVR (SEQ ID NO: 28) (TMHC2_E_V1);
(f) TM 1 comprises the amino acid sequence RLSFSLLLQLVLAIFLLALLIVLLVLLIY (SEQ ID NO: 13) and TM2 comprises the amino acid sequence WLVIVIVALVIIIMVLVLVIIALAVLQMYLVR (SEQ ID NO: 29) (TMHC2_E_V2); and
(g) TM 1 comprises the amino acid sequence RLQLVLAIFLMALLIVLLW (SEQ ID NO: 10) and TM2 comprises the amino acid sequence YLLIVILVLVLVIVALAVTQK (SEQ ID NO: 24) (TMHC2_E_V2);

16. The polypeptide of claim 1, of the general formula X1-TM1-X2-TM2-X3-TM3-X4-TM4, wherein
X3 is a second connecting peptide;
TM3 is a third transmembrane peptide of between 15 and 35 amino acids in length and capable of spanning a biological membrane, wherein (a) the first residue of TM3 is R or K; (b) the last residue of TM3 is W, Y, or L; and (c) at least 60%, 65%, 70%, 75%, 80%, 85%, 90%, or more of the internal residues are hydrophobic;
X4 is an optional third connecting peptide; and
TM4 is an optional fourth transmembrane peptide of between 15 and 35 amino acids in length and capable of spanning a biological membrane, wherein (a) the first residue of TM4 is W, T, Q, or Y; (b) the last residue of TM4 is R or K; and (c) at least 60%, 65%, 70%, 75%, 80%, 85%, 90%, or more of the internal residues are hydrophobic.

17.-20. (canceled)

21. The polypeptide of claim 1, wherein TM1 comprises the amino acid sequence selected from the group consisting of SEQ ID NO:31-34, wherein “X” is any hydrophobic amino acid and Z is any polar amino acid:
>TMHC4, TMHC4_R, TMHC4_E, and TMHC4_R_V3
(SEQ ID NO: 31)
(R/K)ZIXXLLXXAXXXSXXIW(Y/W)
TMHC4_R_V1 and TMHC4_R_V2
(SEQ ID NO: 32)
(R/K)ZIWXXIXXLLXXAXXXSZ(Y/W)
TMHC4, TMHC4_R, TMHC4_E, and TMHC4_R_V3
(SEQ ID NO: 33)
RTIMLLLVFAILLSAIIWY
TMHC4_R_V1 and TMHC4_R_V2
(SEQ ID NO: 34)
RTIWIIIMLLLVFAILLSQY.

22. (canceled)

23. The polypeptide of claim 1, wherein TM2 comprises the amino acid sequence selected from the group consisting of SEQ ID NOS:35-38
(SEQ ID NO: 35)
TLLSXQLLLIAXMLVXIALLLS(R/K)
(SEQ ID NO: 36)
(Q/W/T/Y)QLLLIAXMLVXIALLLS(R/K)
TMYHC4, TMHC4_R, TMHC4_E, and TMHC4_R_V3
(SEQ ID NO: 37)
TLLSMQLLLIALMLVVIALLLSR
TMHC4_R_V1 and TMHC4_R_V2
(SEQ ID NO: 38)
QQLLLIALMLVVIALLLSR
wherein X is any hydrophobic amino acid, wherein “X” is any hydrophobic amino acid.

24. (canceled)

25. The polypeptide of claim 1, wherein TM1 and TM2 comprise a pair selected from the group consisting of:
(a) TM1 comprises the amino acid sequence (R/K)ZIXXLLXXAXXXSXXIW(Y/W) (SEQ ID NO: 31) and TM2 comprises the amino acid sequence TLLSXQLLLIAXMLVXIALLLS(R/K) (SEQ ID NO: 35) (TMHC4)
(b) TM1 comprises the amino acid sequence (R/K)ZIXXLLXXAXXXSXXIW(Y/W) (SEQ ID NO: 31) and TM2 comprises the amino acid sequence TLLSXQLLLIAXMLVXIALLLS(R/K) (SEQ ID NO: 35) (TMHC4_R)
(c) TM1 comprises the amino acid sequence (R/K)ZIXXLLXXAXXXSXXIW(Y/W) (SEQ ID NO: 31) and TM2 comprises the amino acid sequence TLLSXQLLLIAXMLVXIALLLS(R/K) (SEQ ID NO: 35) (TMHC4_E)
(d) TM1 comprises the amino acid sequence (R/K)ZIWXXIXXLLXXAXXXSZ(Y/W) (SEQ ID NO: 32) and TM2 comprises the amino acid sequence (Q/W/T/Y)QLLLIAXMLVXIALLLS(R/K) (SEQ ID NO: 36) (TMHC4_R_V1)
(e) TM1 comprises the amino acid sequence (R/K)ZIWXXIXXLLXXAXXXSZ(Y/W) (SEQ ID NO: 32) and TM2 comprises the amino acid sequence (Q/W/T/Y)QLLLIAXMLVXIALLLS(R/K) (SEQ ID NO: 36) (TMHC4_R_V2)
(f) TM1 comprises the amino acid sequence (R/K)ZIXXLLXXAXXXSXXIW(Y/W) (SEQ ID NO: 31) and TM2 comprises the amino acid sequence TLLSXQLLLIAXMLVXIALLLS(R/K) (SEQ ID NO: 35) (TMHC4_R_V3);
wherein X is any hydrophobic amino acid.

26. The polypeptide of claim 1, wherein TM1 and TM2 comprise a pair selected from the group consisting of:
(a) TM1 comprises the amino acid sequence RTIMLLLVFAILLSAIIWY (SEQ ID NO: 33) and TM2 comprises the amino acid sequence TLLSMQLLLIALMLVVIALLLSR (SEQ ID NO: 37) (TMHC4)
(b) TM1 comprises the amino acid sequence RTIMLLLVFAILLSAIIWY (SEQ ID NO: 33) and TM2 comprises the amino acid sequence TLLSMQLLLIALMLVVIALLLSR (SEQ ID NO: 37) (TMHC4_R)
(c) TM1 comprises the amino acid sequence RTIMLLLVFAILLSAIIWY (SEQ ID NO: 33) and TM2 comprises the amino acid sequence TLLSMQLLLIALMLVVIALLLSR (SEQ ID NO: 37) (TMHC4_E)
(d) TM1 comprises the amino acid sequence RTIWIIIMLLLVFAILLSQY (SEQ ID NO: 34) and TM2 comprises the amino acid sequence QQLLLIALMLVVIALLLSR (SEQ ID NO: 38) (TMHC4_R_V1)
(e) TM1 comprises the amino acid sequence RTIWIIIMLLLVFAILLSQY (SEQ ID NO: 34) and TM2 comprises the amino acid sequence QQLLLIALMLVVIALLLSR (SEQ ID NO: 38) (TMHC4_R_V2); and
(f) TM1 comprises the amino acid sequence RTIMLLLVFAILLSAIIWY (SEQ ID NO: 33) and TM2 comprises the amino acid sequence TLLSMQLLLIALMLVVIALLLSR (SEQ ID NO: 37) (TMHC4_R_V3).

27. The polypeptide of claim 1, wherein TM1 comprises the amino acid sequence (R/K)LLXAVAXLQXLNIXLVX(W/Y/L) (SEQ ID NO: 39), wherein X is any hydrophobic amino acid, or wherein TM1 comprises the amino acid sequence KLLIAVALLOLLNILLVML (SEO ID NO: 40).

28. (canceled)

29. The polypeptide of claim 1, wherein TM2 comprises the amino acid sequence (W/T/Q/Y)MIXXVXXXSXXIVXXAX(R/K) (SEQ ID NO: 41), wherein X is any hydrophobic amino acid, or wherein TM2 comprises the amino acid seguence WMIVIVMFLSLAIVIVALR (SEQ ID NO: 42).

30. (canceled)

31. The polypeptide of claim 1, wherein TM1 comprises the amino acid sequence (R/K)LLXAVAXLQXLNIXLVX(W/Y/L) (SEQ ID NO: 39) and TM2 comprises the amino acid sequence (W/T/Q/Y)MIXXVXXXSXXIVXXAX(R/K) (SEQ ID NO: 41), wherein X is any hydrophobic amino acid.

32. The polypeptide of claim 1, wherein TM1 comprises KLLIAVALLQLLNILLVML (SEQ ID NO: 40) and TM2 comprises the amino acid sequence WMIVIVMFLSLAIVIVALR (SEQ ID NO: 42).

33. The polypeptide of claim 1, of the general formula X1-(TM1-X2-TM2-X3)ₙ, wherein n is 1, 2, 3, or 4.

34.-38. (canceled)

39. The polypeptide of claim 1, comprising the amino acid sequence at least 50%, 55%, 60%, 65%, 70%, 75%, 80%, 85%, 90%, 91%, 92%, 93%, 94%, 95%, 96%, 97%, 98%, 99%, or 100% identical along the length of the amino acid sequence selected from the group consisting of SEQ ID NOS: 43-56.

40.-42. (canceled)

43. A nucleic acid encoding the polypeptide of claim 1.

44.-66. (canceled)
